(19) United States
(12) Patent Application Publication
(10) Pub. No.: US 2024/0218378 A1
(43) Pub. Date: Jul. 4, 2024

(54) MATERIALS AND METHODS FOR PROTEIN PRODUCTION

(71) Applicant: Impossible Foods Inc., Redwood City, CA (US)

(72) Inventors: Biswajoy Roy Chaudhuri, San Mateo, CA (US); Smita Shankar, Millibrae, CA (US)

(21) Appl. No.: 18/609,276

(22) Filed: Mar. 19, 2024

Related U.S. Application Data
(62) Division of application No. 16/858,443, filed on Apr. 24, 2020, now Pat. No. 11,965,167.
(60) Provisional application No. 62/838,770, filed on Apr. 25, 2019.

(51) Int. Cl. C12N 15/52

(52) U.S. Cl. CPC C12N 15/52; C12Y 203/01037

(57) ABSTRACT

This document relates to materials and methods for the production of protein. In one aspect, this document provides a first exogenous nucleic acid construct including a nucleotide sequence encoding an aminolevulinate synthase (ALAS) protein operably linked to a first promoter element, wherein the ALAS includes at least a first heme responsive motif (HRM), and wherein the ALAS includes a mutation in the first HRM, and a second exogenous nucleic acid construct comprising a nucleotide sequence encoding a heme-binding protein, wherein the second exogenous nucleic acid construct including a nucleotide sequence encoding the heme-binding protein is operably linked to the first promoter element or is operably linked to a second promoter element.

### CROSS-REFERENCE TO RELATED APPLICATIONS

This application is a divisional of U.S. application Ser. No. 16/858,443, filed on Apr. 24, 2020, which claims priority to U.S. Provisional Application Ser. No. 62/838,770, filed on Apr. 25, 2019, the contents of each of which are incorporated by reference herein in their entireties.

### SEQUENCE LISTING

This application contains a Sequence Listing that has been submitted electronically as an XML file named “38767-0158002_SL_ST26.xml.” The XML filed, created on March 8, 2024, is 67,846 bytes in size. The material in the XML file is hereby incorporated by reference in its entirety.

### TECHNICAL FIELD

This disclosure generally relates to DNA constructs and methods of using such DNA constructs to genetically engineer cells (e.g., yeast cells (e.g., methylotrophic yeast cells)).

### BACKGROUND

Cells such as Pichia pastoris are commonly used for expression of recombinant proteins. Constructs that can be used to efficiently express one or more proteins in a cell (e.g., fungal cell, such as an Aspergillus cell, a Trichoderma cell, or a yeast cell (e.g., a methylotrophic yeast cell)) are provided herein.

### SUMMARY

This document is based, at least in part, on the identification of mutations in aminolevulinic acid synthase (ALAS) that can confer increased expression of heme-binding proteins. The mutated ALAS proteins described herein can be used for the efficient expression of heme-binding proteins in Pichia, for example.

In one aspect, provided herein is a methylotrophic yeast cell including a first exogenous nucleic acid construct comprising a nucleotide sequence encoding an aminolevulinate synthase (ALAS) protein operably linked to a first promoter element, wherein the ALAS includes a first mutation in a first heme responsive motif (HRM) and a second exogenous nucleic acid construct comprising a nucleotide sequence encoding a heme-binding protein, wherein the second exogenous nucleic acid construct comprising a nucleotide sequence encoding the heme-binding protein is operably linked to the first promoter element or is operably linked to a second promoter element.

Implementations can have one or more of the following features. The methylotrophic yeast cell can be a Pichia cell, a Candida cell, a Hansenula cell, or a Torulopsis cell. The methylotrophic yeast cell can be a Pichia methanolica cell, a Pichia pastoris cell, a Candida boidinii cell, or a Hansenula polymorpha cell. The methylotrophic yeast cell can be a Pichia pastoris cell. The first mutation can be a cysteine to serine mutation. The first mutation can be a cysteine to alanine mutation. The ALAS can include a second mutation in a second HRM. The second mutation can be a cysteine to serine mutation. The second mutation can be a cysteine to alanine mutation. The first exogenous nucleic acid construct can include a nucleic acid sequence having at least 90% sequence identity to the nucleic acid sequence in SEQ ID NO: 28. The first exogenous nucleic acid construct can include a nucleic acid sequence having at least 95% sequence identity to the nucleic acid sequence in SEQ ID NO: 28. The ALAS protein can include an amino acid sequence having at least 90% sequence identity to the amino acid sequence in SEQ ID NO: 29. The ALAS protein can include an amino acid sequence having at least 95% sequence identity to the amino acid sequence in SEQ ID NO: 29. The heme-binding protein can be selected from the group consisting of a globin, a cytochrome, a cytochrome c oxidase, a ligninase, a catalase, and a peroxidase. The heme-binding protein can be selected from the group consisting of an androglobin, a chlorocruorin, a cytoglobin, an erythrocruorin, a flavohemoglobin, a globin E, a globin X, a globin Y, a hemoglobin, a histoglobin, a leghemoglobin, a myoglobin, a neuroglobin, a non-symbiotic hemoglobin, a protoglobin, and a truncated hemoglobin. The heme-binding protein can be a non-symbiotic hemoglobin. The heme-binding protein can be a leghemoglobin. The heme-binding protein can include an amino acid sequence having at least 90% sequence identity to an amino acid sequence in any one of SEQ ID NOs: 1-27. The methylotrophic yeast cell can further include a third nucleic acid construct comprising a nucleotide sequence encoding a transcription factor, wherein the third nucleic acid construct is operably linked to the first promoter element, the second promoter element, or a third promoter element. The first promoter element can include a recognition sequence for the transcription factor. The second exogenous nucleic acid construct can be operably linked to a second promoter element, and wherein the second promoter element includes a recognition sequence for the transcription factor. The third nucleic acid construct can be operably linked to the third promoter element, and wherein the third promoter element includes a recognition sequence for the transcription factor. The methylotrophic yeast cell can further include a fourth nucleic acid construct comprising a nucleotide sequence encoding a protein involved in heme biosynthesis, wherein the fourth nucleic acid construct is operably linked to the first promoter element, the second promoter element, the third promoter element, or a fourth promoter element. The protein involved in heme biosynthesis can be selected from the group consisting of ALA dehydratase, porphobilinogen deaminase, UPG III synthase, UPG III decarboxylase, CPG oxidase, PPG oxidase, and ferrochelatase. The first exogenous nucleic acid construct can be a heterologous nucleic acid construct. The second exogenous nucleic acid construct can be a heterologous nucleic acid construct. The heme-binding protein can be an exogenous heme-binding protein. The heme-binding protein can be a heterologous heme-binding protein.

In another aspect, provided herein is a method of producing a heme-binding protein in a methylotrophic yeast cell including expressing a first exogenous nucleic acid construct comprising a nucleotide sequence encoding an aminolevulinate synthase (ALAS) protein operably linked to a first promoter element, wherein the ALAS includes a first mutation in a first heme responsive motif (HRM) and expressing a second exogenous nucleic acid construct comprising a nucleotide sequence encoding a heme-binding protein, wherein the second exogenous nucleic acid construct comprising a nucleotide sequence encoding the heme-binding protein is operably linked to the first promoter element or is operably linked to a second promoter element.

Implementations can have one or more of the following features. The ALAS can include a second mutation in a second HRM. The method can produce the heme-binding protein in a titer that is at least 5% greater than a corresponding method lacking the first exogenous nucleic acid construct. The method can produce the heme-binding protein in a titer that is at least 10% greater than a corresponding method lacking the first exogenous nucleic acid construct. The method can produce the heme-binding protein in a titer that is at least 15% greater than a corresponding method lacking the first exogenous nucleic acid construct. The method can produce the heme-binding protein in a titer that is at least 20% greater than a corresponding method lacking the first exogenous nucleic acid construct. The method can produce the heme-binding protein in a titer that is at least 5% greater than a corresponding method lacking the first mutation. The method can produce the heme-binding protein in a titer that is at least 10% greater than a corresponding method lacking the first mutation. The method can produce the heme-binding protein in a titer that is at least 15% greater than a corresponding method lacking the first mutation. The method can produce the heme-binding protein in a titer that is at least 20% greater than a corresponding method lacking the first mutation. The method can produce the heme-binding protein in a titer that is at least 5% greater than a corresponding method lacking the first mutation and the second mutation. The method can produce the heme-binding protein in a titer that is at least 10% greater than a corresponding method lacking the first mutation and the second mutation. The method can produce the heme-binding protein in a titer that is at least 15% greater than a corresponding method lacking the first mutation and the second mutation. The method can produce the heme-binding protein in a titer that is at least 20% greater than a corresponding method lacking the first mutation and the second mutation. The method can be carried out in the absence of added methanol. The methylotrophic yeast cell can be a Pichia cell, a Candida cell, a Hansenula cell, or a Torulopsis cell. The methylotrophic yeast cell can be a Pichia methanolica cell, a Pichia pastoris cell, a Candida boidinii cell, or a Hansenula polymorpha cell. The methylotrophic yeast cell can be a Pichia pastoris cell. The first mutation can be a cysteine to serine mutation. The first mutation can be a cysteine to alanine mutation. The second mutation can be a cysteine to serine mutation. The second mutation can be a cysteine to alanine mutation. The first exogenous nucleic acid construct can include a nucleic acid sequence having at least 90% sequence identity to the nucleic acid sequence in SEQ ID NO: 28. The first exogenous nucleic acid construct can include a nucleic acid sequence having at least 95% sequence identity to the nucleic acid sequence in SEQ ID NO: 28. The ALAS protein can include an amino acid sequence having at least 90% sequence identity to the amino acid sequence in SEQ ID NO: 29. The ALAS protein can include an amino acid sequence having at least 95% sequence identity to the amino acid sequence in SEQ ID NO: 29. The heme-binding protein can be selected from the group consisting of a globin, a cytochrome, a cytochrome c oxidase, a ligninase, a catalase, and a peroxidase. The heme-binding protein can be selected from the group consisting of an androglobin, a chlorocruorin, a cytoglobin, an erythrocruorin, a flavohemoglobin, a globin E, a globin X, a globin Y, a hemoglobin, a histoglobin, a leghemoglobin, a myoglobin, a neuroglobin, a non-symbiotic hemoglobin, a protoglobin, and a truncated hemoglobin. The heme-binding protein can be a non-symbiotic hemoglobin. The heme-binding protein can be a leghemoglobin. The heterologous heme-binding protein can include an amino acid sequence having at least 90% sequence identity to an amino acid sequence in any one of SEQ ID NOs: 1-27. The method can further include expressing a third nucleic acid construct comprising a nucleotide sequence encoding a transcription factor, wherein the third nucleic acid construct is operably linked to the first promoter element, the second promoter element, or a third promoter element. The first promoter element can include a recognition sequence for the transcription factor. The second exogenous nucleic acid construct can be operably linked to a second promoter element, and wherein the second promoter element includes a recognition sequence for the transcription factor. The third nucleic acid construct can be operably linked to the third promoter element, and wherein the third promoter element includes a recognition sequence for the transcription factor. The method can further include expressing a fourth nucleic acid construct comprising a nucleotide sequence encoding a protein involved in heme biosynthesis, wherein the fourth nucleic acid construct is operably linked to the first promoter element, the second promoter element, the third promoter element, or a fourth promoter element. The protein involved in heme biosynthesis can be selected from the group consisting of ALA dehydratase, porphobilinogen deaminase, UPG III synthase, UPG III decarboxylase, CPG oxidase, PPG oxidase, and ferrochelatase. The first exogenous nucleic acid construct can be a heterologous nucleic acid construct. The second exogenous nucleic acid construct can be a heterologous nucleic acid construct. The heme-binding protein can be an exogenous heme-binding protein. The heme-binding protein can be a heterologous heme-binding protein.

In another aspect, provided herein is a Pichia pastoris cell including a first exogenous nucleic acid construct comprising a nucleotide sequence encoding an aminolevulinate synthase (ALAS) protein operably linked to a first promoter element, wherein the ALAS includes a first mutation in a first heme responsive motif (HRM) and a second mutation in a second HRM and a second exogenous nucleic acid construct comprising a nucleotide sequence encoding leghemoglobin, wherein the second exogenous nucleic acid construct comprising a nucleotide sequence encoding leghemoglobin is operably linked to the first promoter element, or the second exogenous nucleic acid construct comprising a nucleotide sequence encoding leghemoglobin is operably linked to a second promoter element.

Implementations can include one or more of the following features. The first mutation can be a cysteine to serine mutation. The second mutation can be a cysteine to serine mutation.

In another aspect, provided herein is a method of producing leghemoglobin, the method including expressing a first exogenous nucleic acid construct comprising a nucleotide sequence encoding an aminolevulinate synthase (ALAS) protein operably linked to a first promoter clement, wherein the ALAS includes a first mutation in a first heme responsive motif (HRM) and a second mutation in a second HRM and expressing a second exogenous nucleic acid construct comprising a nucleotide sequence encoding leghemoglobin, wherein the second exogenous nucleic acid construct comprising a nucleotide sequence encoding leghemoglobin is operably linked to the first promoter element, or the second exogenous nucleic acid construct comprising a nucleotide sequence encoding leghemoglobin is operably linked to a second promoter element.

Implementations can have one or more of the following features. The method can produce the leghemoglobin in a titer that is at least 5% greater than a corresponding method lacking the first exogenous nucleic acid construct. The method can produce the leghemoglobin in a titer that is at least 10% greater than a corresponding method lacking the first exogenous nucleic acid construct. The method can produce the leghemoglobin in a titer that is at least 15% greater than a corresponding method lacking the first exogenous nucleic acid construct. The method can produce the leghemoglobin in a titer that is at least 20% greater than a corresponding method lacking the first exogenous nucleic acid construct. The method can produce the leghemoglobin in a titer that is at least 5% greater than a corresponding method lacking the first mutation and the second mutation. The method can produce the leghemoglobin in a titer that is at least 10% greater than a corresponding method lacking the first mutation and the second mutation. The method can produce the leghemoglobin in a titer that is at least 15% greater than a corresponding method lacking the first mutation and the second mutation. The method can produce the leghemoglobin in a titer that is at least 20% greater than a corresponding method lacking the first mutation and the second mutation. The method can be carried out in the absence of added methanol.

In another aspect, provided herein is a Pichia pastoris cell including a first exogenous nucleic acid construct comprising a nucleotide sequence encoding a protein with at least 90% sequence identity to SEQ ID NO: 29, wherein the nucleic acid encodes a serine residue in a position corresponding to position 12 of SEQ ID NO: 29, and wherein the nucleic acid encodes a serine residue in a position corresponding to position 39 of SEQ ID NO: 29, operably linked to a first promoter element.

Implementations can include one or more of the following features. The Pichia pastoris cell can further include a second exogenous nucleic acid construct comprising a nucleotide sequence encoding a protein with at least 90% sequence identity to any of SEQ ID NOs: 1-27, wherein the second exogenous nucleic acid construct is operably linked to the first promoter element, or the second exogenous nucleic acid construct is operably linked to a second promoter element. The Pichia pastoris cell can further include a second exogenous nucleic acid construct comprising a nucleotide sequence encoding a protein with at least 90% sequence identity to SEQ ID NO: 4, wherein the second exogenous nucleic acid construct is operably linked to the first promoter element, or the second exogenous nucleic acid construct is operably linked to a second promoter element.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims. The word “comprising” in the claims may be replaced by “consisting essentially of” or with “consisting of,” according to standard practice in patent law.

### DESCRIPTION OF THE DRAWINGS

FIG. 1 provides a schematic depicting the steps involved in the heme biosynthesis pathway.

FIG. 2 provides a structure of Heme b.

FIG. 3 provides a structure of hemin.

FIG. 4 provides the sequences of exemplary heme-binding proteins (SEQ ID NOs: 1-27).

FIG. 5 provides an exemplary construct diagram of a mutated ALAS.

FIG. 6 provides the nucleic acid sequence (SEQ ID NO: 28) and amino acid sequence (SEQ ID NO: 29) of wild-type ALAS.

FIG. 7 provides the nucleic acid sequence (SEQ ID NO: 30) and amino acid sequence (SEQ ID NO: 31) of an exemplary mutant ALAS.

FIG. 8 provides the sequences of SEQ ID NOs: 32-66.

### DETAILED DESCRIPTION

This document is related to materials and methods for protein production. In particular, this document is related to materials and method for the production of heme and heme-binding proteins.

Methylotrophic yeast, such as Pichia pastoris, are commonly used to produce recombinant proteins. Pichia strains are typically able to grow using methanol as the sole carbon source. It will be understood that Pichia pastoris has been reclassified as Komagataella species, such as Komagataella phaffii, Komagataella pastoris, or Komagataella pseudopastoris, though the term ‘Pichia pastoris’ is still in use and may refer to any appropriate Komagataella species. Commonly, laboratory strains of P. pastoris are Komagataella phaffii.

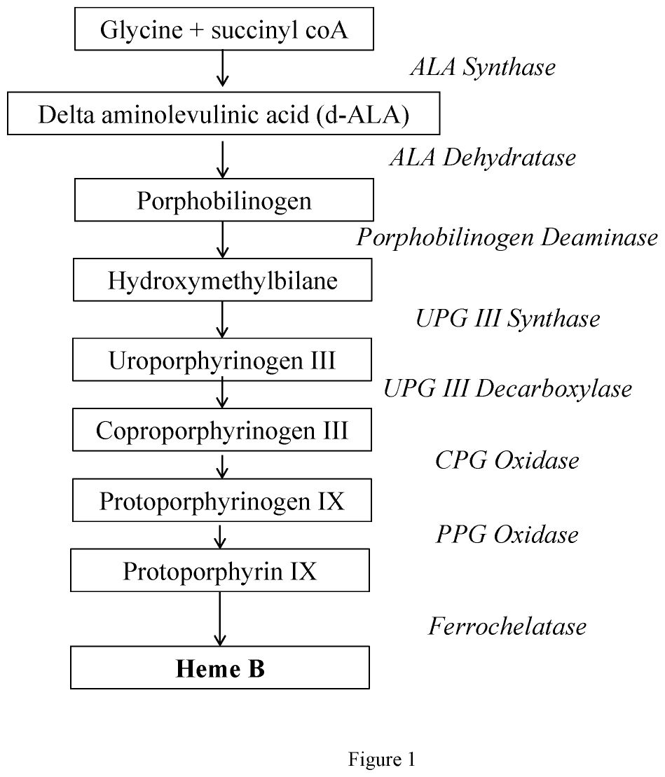

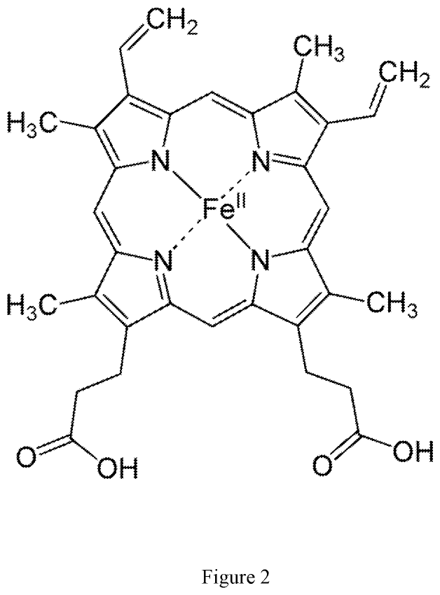

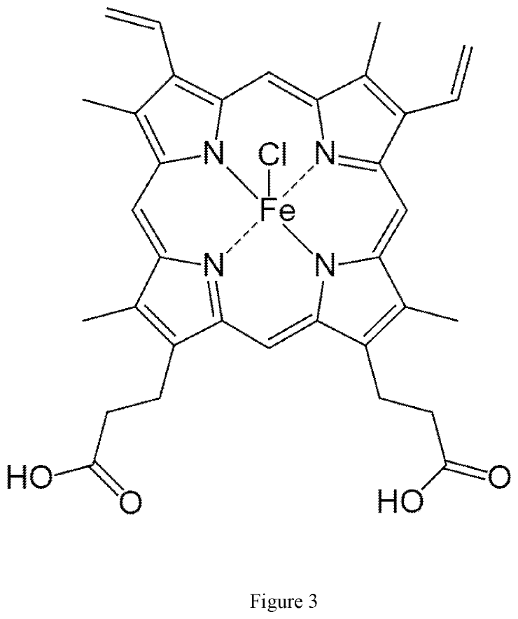

Proteins that bind heme include cytochromes, catalase, myoglobin and hemoglobin among others. Aminolevulinic acid synthase (ALAS) is an enzyme (EC 2.3.1.37) that catalyzes the first step in heme biosynthesis (See FIG. 1 for a diagram of a Heme b biosynthesis pathway), catalyzing the transformation of glycine and succinyl-CoA to aminolevulinic acid. ALAS is translocated to the mitochondria for this step of heme biosynthesis. Aminolevulinic acid is transformed by other enzymes (e.g., ALA dehydratase (ALAD), porphobilinogen deaminase (PBGD), uroporphyrinogen III synthase (UPG3S), uroporphyrinogen III decarboxylase (UPG3D), coprotoporphyrinogen oxidase (COPROX), protoporphyrinogen IX oxidase (PROTOX), and/or ferrochelatase (FC)) to Heme b (FIG. 2). In some cases, the action of ALAS can be rate-limiting for the heme biosynthesis pathway. Other hemes (e.g., Heme o, Heme a, Heme c) can be made through the action of enzymes on Heme b. Hemin (FIG. 3) is a complex of protoporphyrin IX (e.g., the protoporphyrin of Heme b) with a ferric (+3 oxidation state) iron and a chloride ligand.

As used herein, “intermediate of the heme biosynthesis pathway” refers to one or more of: delta aminolevulinic acid (d-ALA), porphobilinogen, hydroxymethylbilane, uroporphyrinogen III, coproporphyrinogen III, protoporphyrinogen IX, or protoporphyrin IX. In some embodiments, an intermediate of the heme biosynthesis pathway can be selected from the group consisting of delta aminolevulinic acid (d-ALA), porphobilinogen, hydroxymethylbilane, uroporphyrinogen III, coproporphyrinogen III, protoporphyrinogen IX, and protoporphyrin IX. In some embodiments, an intermediate of the heme biosynthesis pathway can be selected from the group consisting of porphobilinogen, hydroxymethylbilane, uroporphyrinogen III, coproporphyrinogen III, protoporphyrinogen IX, or protoporphyrin IX.

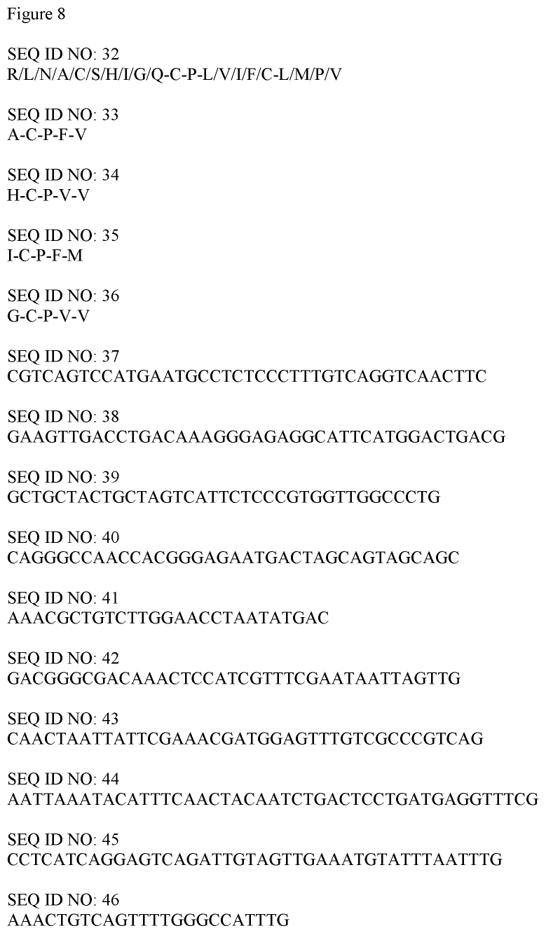

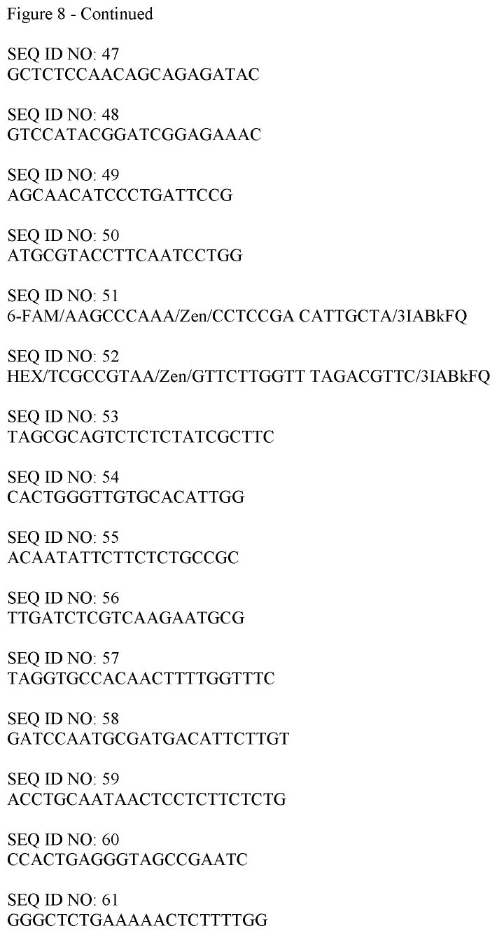

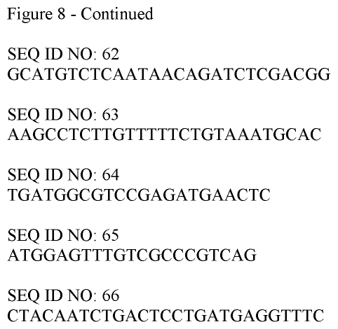

Translocation of some ALAS proteins to the mitochondria can be affected by one or more heme regulatory motifs (HRMs; sometimes also called heme responsive motifs) contained within the ALAS protein. Many HRMs include a C-P motif, and the cysteine is often an axial ligand for heme. In some embodiments, a HRM has a sequence of R/L/N/A/C/S/H/I/G/Q-C-P-L/V/I/F/C-L/M/P/V (SEQ ID NO: 32) (e.g., a HRM can have a sequence of A-C-P-F-V (SEQ ID NO: 33), H-C-P-V-V (SEQ ID NO: 34), I-C-P-F-M (SEQ ID NO: 35), or G-C-P-V-V (SEQ ID NO: 36); sec, e.g., FIG. 8). Some organisms have 3 HRMs in their ALAS sequence, while other organisms (e.g., a methylotrophic yeast such as Pichia pastoris) have two. In general, a HRM can be called HRM1, HRM2, HRM3, and so forth, depending on where the HRM occurs in the protein sequence, e.g., the first-occurring HRM in the protein sequence (read as N-terminus to C-terminus) would be called HRM1. Mutation of the cysteine (e.g., to serine or alanine) in the HRMs can increase the translocation of ALAS to the mitochondria (see, e.g., González-Domínguez, et al., Yeast. 2001 Jan 15;18(1):41-8. (PubMed ID (PMID) 11124700); Munakata et al., J Biochem. 2004 Aug; 136(2):233-8. (PMID 15496594); Dailey et al, Biochem J. 2005 Mar 1;386(Pt 2):381-6. (PMID 15482256)), each of which is herein incorporated by reference in its entirety). Without being bound by any particular theory, it is believed that heme binding to the HRMs of wild type ALAS can inhibit the translocation of ALAS to the mitochondria in a negative feedback manner. Heme is also believed to be involved in the degradation of some ALAS proteins (see, e.g., Kubota, et al., J Biol Chem. 2016 Sep 23;291(39):20516-29. doi: 10.1074/jbc.M116.719161. Epub 2016 Aug 5. (PMID 27496948), incorporated by reference herein in its entirety).

It will be appreciated that a “first HRM” can be an HRM in any part of a protein sequence; a “first HRM” can be, but is not necessarily, the first-occurring HRM (HRM1) in a protein sequence. In some embodiments, a first HRM is HRM1, and a second HRM is HRM2. In some embodiments, a first HRM is HRM1, and a second HRM is HRM3. In some embodiments, a first HRM is HRM2, and a second HRM is HRM1. In some embodiments, a first HRM is HRM2, and a second HRM is HRM3. In some embodiments, a first HRM is HRM3, and a second HRM is HRM1. In some embodiments, a first HRM is HRM3, and a second HRM is HRM2.

In some embodiments, a mutation in a nucleic acid can be an insertion, a deletion, or a substitution. In some embodiments, a mutation in a nucleic acid can be a substitution (c.g., a guanosine to cytosine mutation). In some embodiments, a substitution in a coding sequence (c.g., encoding a protein) can be a silent mutation (e.g., the same amino acid is encoded). In some embodiments, a substitution in a coding sequence can be a nonsynonymous mutation (c.g., a missense mutation or a nonsense mutation). In some embodiments, a substitution in a coding sequence can be a missense mutation (e.g., a different amino acid is encoded). In some embodiments, a substitution in a coding sequence can be nonsense mutation (e.g., a premature stop codon is encoded). In some embodiments, a mutation in a nucleic acid can be can be a deletion. It will be understood that mutations can be used to alter an endogenous nucleic acid, using, for example, CRISPR, TALEN, and/or Zinc-finger nucleases.

In some embodiments, a mutation in a protein sequence can be an insertion, a deletion, or a substitution. It will be understood that a mutation in a nucleic acid that encodes a protein can cause a mutation in a protein sequence. In some embodiments, a mutation in a protein sequence is a substitution (e.g., a cysteine to serine mutation, a cysteine to alanine mutation, a cysteine to valine mutation, a cysteine to leucine mutation, a cysteine to isoleucine mutation, a cysteine to glycine mutation, a cysteine to phenylalanine mutation, a cysteine to threonine mutation, a cysteine to methionine mutation, a cysteine to tryptophan mutation, a cysteine to tyrosine mutation, a cysteine to asparagine mutation, a cysteine to glutamine mutation, a cysteine to proline mutation, a cysteine to arginine mutation, a cysteine to histidine mutation, a cysteine to lysine mutation, a cysteine to aspartic acid mutation, or a cysteine to glutamic acid mutation). In some embodiments, a mutation in a protein sequence can be a deletion.

In some embodiments, an ALAS protein as described herein can include a mutation in at least one HRM; for example, a first HRM, a second HRM, a third HRM, a fourth HRM, a fifth HRM, and so forth.

In some embodiments, an ALAS protein as described herein can include a mutation in at least one HRM (e.g., a first HRM). In some embodiments, an ALAS protein as described herein can include a mutation in a single HRM (e.g., a first HRM or a second HRM). In some embodiments, an ALAS protein as described herein can include a mutation in a cysteine of at least one HRM (e.g., a first HRM). In some embodiments, an ALAS protein as described herein can include a mutation in a cysteine in a single HRM (e.g., a first HRM or a second HRM).

In some embodiments, an ALAS protein as described herein can include a mutation in at least two HRMs (e.g., a first HRM and a second HRM). In some embodiments, an ALAS protein as described herein can include a mutation in each of two HRMs (e.g., a first HRM and a second HRM). In some embodiments, the mutation in the first HRM and the mutation in the second HRM can be the same (e.g., both cysteine to serine or alanine mutations). In some embodiments, the mutation in the first HRM and the mutation in the second HRM can be different (e.g., one is a cysteine to serine mutation and one is a cysteine to alanine mutation). In some embodiments, an ALAS protein as described herein can include a mutation in a cysteine of at least two HRMs (e.g., a first HRM and a second HRM). In some embodiments, an ALAS protein as described herein can include a mutation in a cysteine in each of two HRMs (e.g., a first HRM and a second HRM). In some embodiments, the mutation in the first HRM and the mutation in the second HRM can be the same (e.g., both cysteine to serine or alanine mutations). In some embodiments, the mutation in the first HRM and the mutation in the second HRM can be different (e.g., one is a cysteine to serine mutation and one is a cysteine to alanine mutation).

In some embodiments, an ALAS protein as described herein can include a mutation in at least three HRMs (e.g., a first HRM, a second HRM, and a third HRM). In some embodiments, an ALAS protein as described herein can include a mutation in each of three HRMs (e.g., a first HRM, a second HRM, and a third HRM). In some embodiments, an ALAS protein as described herein can include a mutation in a cysteine at least three HRMs (e.g., a first HRM, a second HRM, and a third HRM). In some embodiments, an ALAS protein as described herein can include a mutation in a cysteine in each of three HRMs (e.g., a first HRM, a second HRM, and a third HRM). In some embodiments, the mutation in each of the first HRM, the second HRM, and the third HRM can be the same (e.g., both cysteine to serine or alanine mutations). In some embodiments, the mutation in one or two of the three HRMs can be different.

In some embodiments, a substitution (a mutation to a different amino acid) can be a substitution for an amino acid (e.g., a cysteine) in a HRM. In some embodiments, a mutation in a first HRM can be a substitution for an amino acid (e.g., a cysteine) in the first HRM. In some embodiments, a mutation in a second HRM can be a substitution for an amino acid (e.g., a cysteine) in the second HRM. In some embodiments, a mutation in a third HRM can be a substitution for an amino acid (e.g., a cysteine) in the third HRM. A substitution can be any appropriate substitution. In some embodiments, a different amino acid can be selected from group consisting of arginine, histidine, lysine, serine, threonine, asparagine, glutamine, selenocysteine, glycine, proline, alanine, isoleucine, leucine, methionine, phenylalanine, tryptophan, tyrosine, and valine. In some embodiments, a different amino acid can be selected from group consisting of arginine, histidine, lysine, serine, threonine, asparagine, glutamine, selenocysteine, glycine, proline, alanine, isoleucine, leucine, methionine, aspartic acid, glutamic acid, phenylalanine, tryptophan, tyrosine, and valine. In some embodiments, a different amino acid is selected from a nonpolar aliphatic amino acid (e.g., glycine, proline, alanine, isoleucine, leucine, methionine, or valine), an aromatic amino acid (e.g., phenylalanine, tryptophan, or tyrosine), a polar uncharged amino acid (e.g., serine, threonine, asparagine, or glutamine), or a positively charged amino acid (arginine, histidine, or lysine). In some embodiments, the different amino acid is serine. In some embodiments, the different amino acid is alanine. In some embodiments, the different amino acid is phenylalanine. In some embodiments, the different amino acid is aspartic acid. In some embodiments, the different amino acid is histidine.

Surprisingly, when an ALAS protein with a cysteine to serine mutation in each of its two HRMs is co-expressed with an exogenous heme-binding protein, the titer of the exogenous heme-binding protein can increase significantly.

Generally, a “titer” is the measurement of the amount of a substance in solution. As used hercin, the “titer” of a heme-binding protein refers to the overall amount of the polypeptide, whether or not it is bound to heme, unless otherwise specified. The titer of a protein can be measured by suitable method, such as high-performance liquid chromatography (HPLC or UPLC), liquid chromatography-mass spectrometry (LC-MS), an enzyme-linked immunosorbent assay (ELISA), enzyme activity measurements, iron measurement techniques such as atomic absorption spectroscopy, LC-MS, or ultraviolet and/or visible light spectroscopy.

Mutations in ALAS, such as those described herein, can be used to increase heme production. In some embodiments, the titer of a heme-binding protein can be increased. In some embodiments, the titer of a leghemoglobin (LegH) can be increased. The materials and methods of this disclosure can be useful to increase the production of heme.

The materials and methods described herein can have applications in many industries. For example, heme proteins can be used in food. As another example, heme proteins (e.g., hemoglobins, cytochrome P450s) can be produced for research purposes (e.g., to study drug metabolism). Heme proteins can also be used in industry (e.g., catalases, laccases, and/or peroxidases for uses such as washing detergents, pulp bleaching, lignin degradation). Another potential application for heme proteins is in biocatalysis (e.g. cytochrome P450s, lipoxygenases, and/or laccases can be used for the production of pharmaceutical and commodity chemicals). Some heme proteins can be used as therapeutic agents (e.g., nitric oxide synthases or as part of a blood replacement). In some cases, heme proteins can be used for electronic purposes (e.g. for the production of renewable electricity). As another example, heme therapy can be a treatment option for patients with acute porphyrias, a group of eight genetic diseases that result from inability to produce heme.

Nucleic acid constructs (sometimes also called nucleic acid molecules) are provided herein that allow for genetically engineering a cell (e.g., fungal cell, such as an Aspergillus cell, a Trichoderma cell, or a yeast cell (e.g., a methylotrophic yeast cell)) to produce a mutant ALAS. In addition, nucleic acid constructs are provided herein that allow for genetically engineering a cell (e.g., fungal cell, such as an Aspergillus cell, a Trichoderma cell, or a yeast cell (e.g., a methylotrophic yeast cell)) to increase the expression of a heme-binding protein. In some embodiments, nucleic acid constructs are provided herein that allow for genetically engineering a cell (e.g., fungal cell, such as an Aspergillus cell, a Trichoderma cell, or a yeast cell (e.g., a methylotrophic yeast cell)) to increase the expression of a heme-binding protein from an inducible promoter in the absence of the inducing molecule.

Nucleic acid constructs are provided herein that allow for genetically engineering a cell. A cell can be any appropriate cell. For example, a cell can be a bacterial cell (e.g., an E. coli cell, a B. subtilis cell, or a Lactococcus lactis cell), a fungal cell, an algal cell, a plant cell, an insect cell, or a mammalian cell. In some embodiments, a cell can be a fungal cell. In some embodiments, a cell can be a filamentous fungus cell. In some embodiments, a cell can be an Aspergillus or Trichoderma cell. In some embodiments, a cell can be a yeast cell. Non-limiting examples of yeast cells include Pichia (e.g., Pichia methanolica, Pichia pastoris), Candida (e.g., (Candida boidinii) cells, Hansenula (e.g., Hansemila polymorpha) cells, Torulopsis cells, and Sacharomyces (e.g., Sacharomyces cerevisae) cells. In some embodiments, a cell can be a methylotrophic yeast cell. Non-limiting examples of methylotrophic yeast cells include Pichia cells, Candida cells, Hansenula cells, and Torulopsis cells. In some embodiments, a cell can be a Pichia cell or a Sacharomyces cell. In some embodiments, a cell is a eukaryotic cell. In some embodiments, a cell is a fungal cell. In some embodiments, a cell is a plant cell. In some embodiments, a cell is an algal cell. In some embodiments, a cell is a yeast cell. In some embodiments, a cell is a Saccharomyces cerevisiae cell. In some embodiments, a cell is a methylotrophic yeast cell. While the methods are exemplified herein using a Pichia species (e.g., P. pastoris), other cells can be used, such as other species of the Pichia genus or species from any of the Candida, Hansenula, Pichia, and Torulopsis genera. Non-limiting examples of species of methylotrophic yeast include Pichia methanolica, Pichia pastoris, Candida boidinii, and Hansenula polymorpha.

Accordingly, in one aspect, this document provides materials and methods for expressing protein. In some embodiments, this document provides a cell (e.g., fungal cell, such as an Aspergillus cell, a Trichoderma cell, or a yeast cell (e.g., a methylotrophic yeast cell)) including a first nucleic acid construct comprising a nucleotide sequence encoding an ALAS protein. In some embodiments, the first nucleic acid encodes an ALAS protein operably linked to a first promoter element. In some embodiments, an ALAS protein includes at least 1 HRM (c.g., 1, 2, 3, or more HRMs). In some embodiments, an ALAS protein includes at least 2 HRMs (e.g., 2, 3 or more HRMs). In some embodiments, an ALAS protein includes at least 3 HRMs (e.g., 3 or more HRMs). In some embodiments, the ALAS protein includes a mutation in a first HRM (sometimes also called “a first mutation in a first HRM”). In some embodiments, the ALAS protein includes a mutation in a second HRM (sometimes also called “a second mutation in a second HRM”). In some embodiments, the ALAS protein includes a mutation in a first HRM and a mutation in a second HRM. In some embodiments, the first HRM is HRM1. In some embodiments, the second HRM is HRM2. In some embodiments, the ALAS protein includes a mutation in a third HRM (sometimes also called “a third mutation in a third HRM”). In some embodiments, the ALAS protein includes a mutation in a first HRM, a mutation in a second HRM, and a mutation in a third HRM.

In some embodiments, the mutation in the first HRM is a substitution. In some embodiments, the mutation in the first HRM is a cysteine substitution. In some embodiments, the mutation in the first HRM is a cysteine to serine mutation. In some embodiments, the mutation in the first HRM is a cysteine to alanine mutation. In some embodiments, the mutation in the first HRM is a cysteine to phenylalanine mutation. In some embodiments, the mutation in the first HRM is a cysteine to aspartic acid mutation. In some embodiments, the mutation in the first HRM is a cysteine to histidine mutation.

In some embodiments, the mutation in the second HRM is a substitution. In some embodiments, the mutation in the second HRM is a cysteine substitution. In some embodiments, the mutation in the second HRM is a cysteine to serine mutation. In some embodiments, the mutation in the second HRM is a cysteine to alanine mutation. In some embodiments, the mutation in the second HRM is a cysteine to phenylalanine mutation. In some embodiments, the mutation in the second HRM is a cysteine to aspartic acid mutation. In some embodiments, the mutation in the second HRM is a cysteine to histidine mutation.

In some embodiments, the mutation in the third HRM is a substitution. In some embodiments, the mutation in the third HRM is a cysteine substitution. In some embodiments, the mutation in the third HRM is a cysteine to serine mutation. In some embodiments, the mutation in the third HRM is a cysteine to alanine mutation. In some embodiments, the mutation in the third HRM is a cysteine to phenylalanine mutation. In some embodiments, the mutation in the third HRM is a cysteine to aspartic acid mutation. In some embodiments, the mutation in the third HRM is a cysteine to histidine mutation.

In some embodiments, the mutation in the first HRM corresponds to a mutation in residue 12 in SEQ ID NO: 29. In some embodiments, the mutation in the second HRM corresponds to a mutation in residue 39 in SEQ ID NO: 29. In some embodiments, the mutation in the first HRM is a cysteine to serine mutation corresponding to a cysteine to serine mutation in residue 12 in SEQ ID NO: 29. In some embodiments, the mutation in the first HRM is a cysteine to alanine mutation corresponding to a cysteine to alanine mutation in residue 12 in SEQ ID NO: 29. In some embodiments, the mutation in the first HRM is a cysteine to phenylalanine mutation corresponding to a cysteine to phenylalanine mutation in residue 12 in SEQ ID NO: 29. In some embodiments, the mutation in the first HRM is a cysteine to histidine mutation corresponding to a cysteine to histidine mutation in residue 12 in SEQ ID NO: 29. In some embodiments, the mutation in the second HRM is a cysteine to serine mutation corresponding to a cysteine to serine mutation in residue 39 in SEQ ID NO: 29. In some embodiments, the mutation in the second HRM is a cysteine to alanine mutation corresponding to a cysteine to alanine mutation in residue 39 in SEQ ID NO: 29. In some embodiments, the mutation in the second HRM is a cysteine to phenylalanine mutation corresponding to a cysteine to phenylalanine mutation in residue 39 in SEQ ID NO: 29. In some embodiments, the mutation in the second HRM is a cysteine to histidine mutation corresponding to a cysteine to histidine mutation in residue 39 in SEQ ID NO: 29.

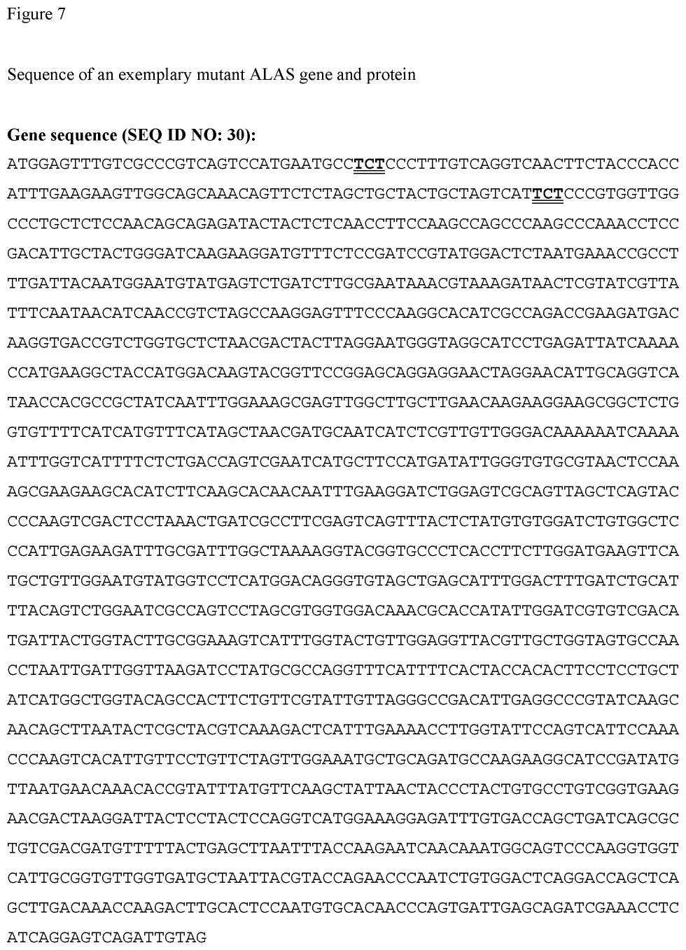

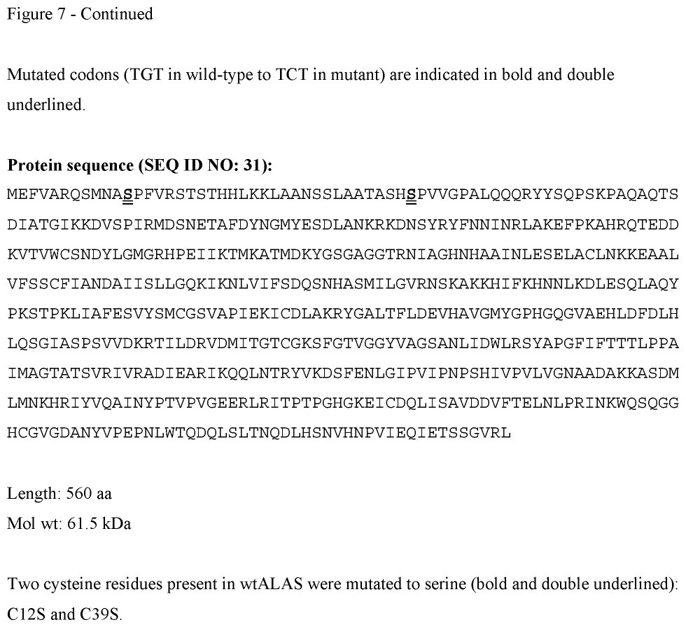

In some embodiments, the first nucleic acid includes a mutation corresponding to a guanosine to cytosine mutation at nucleotide position 35 in SEQ ID NO: 28. In some embodiments, the first nucleic acid includes a mutation corresponding to a thymine to guanosine mutation at nucleotide position 34 in SEQ ID NO: 28 and a guanosine to cytosine mutation at nucleotide position 35 in SEQ ID NO: 28. In some embodiments, the first nucleic acid includes a mutation corresponding to a guanosine to cytosine mutation at nucleotide position 116 in SEQ ID NO: 28. In some embodiments, the first nucleic acid includes a mutation corresponding to a thymine to guanosine mutation in nucleotide position 115 in SEQ ID NO: 28 and a guanosine to cytosine mutation at nucleotide position 116 in SEQ ID NO: 28. It will be appreciated that cysteine to serine mutations can be accomplished by mutations at nucleotides other than those specifically disclosed herein. It will be appreciated that cysteine to alanine mutations can be accomplished by mutations at nucleotides other than those specifically disclosed herein. In some embodiments, the first nucleic acid construct includes SEQ ID NO: 30. In some embodiments, the ALAS protein has the sequence of SEQ ID NO: 31. (See, e.g., FIG. 7).

As used herein, “operably linked” means that a promoter or other expression element(s) are positioned relative to a nucleic acid coding sequence in such a way as to direct or regulate expression of the coding sequence (e.g., in-frame).

A “corresponding” amino acid position (or substitution) in a protein sequence different from a reference protein sequence (e.g., in the ALAS protein sequence of a different organism compared to a reference ALAS protein sequence, such as SEQ ID NO: 29) can be identified by performing a sequence alignment between the protein sequences of interest. It will be understood that in some cases, a gap exists in a protein alignment. Similarly, a “corresponding” nucleic acid position (or substitution) in a nucleic acid sequence different from a reference nucleic acid sequence (e.g., in the ALAS nucleic acid sequence of a different organism compared to a reference ALAS nucleic acid sequence, such as SEQ ID NO: 28) can be identified by performing a sequence alignment between the nucleic acid sequences of interest. It will be understood that in some cases, a gap exists in a nucleic acid alignment. As used herein, a nucleotide or amino acid position “relative to” a reference sequence can be the corresponding nucleotide or amino acid position in a reference sequence.

Nucleic acid molecules used in the methods described herein are typically DNA, but RNA molecules can be used under the appropriate circumstances. As used herein, “exogenous” refers to any nucleic acid sequence that is introduced into a cell from, for example, the same or a different organism or a nucleic acid generated synthetically (e.g., a codon-optimized nucleic acid sequence). For example, an exogenous nucleic acid can be a nucleic acid from one microorganism (e.g., one genus or species of methylotrophic yeast) that is introduced into a different genus or species of methylotrophic yeast; however, an exogenous nucleic acid also can be a nucleic acid from a methylotrophic yeast that is introduced recombinantly into a methylotrophic yeast as an additional copy despite the presence of a corresponding native nucleic acid sequence, or a nucleic acid from a methylotrophic yeast that is introduced recombinantly into a methylotrophic yeast containing one or more mutations, insertions, or deletions compared to the sequence native to the methylotrophic yeast. For example, P. pastoris contains an endogenous nucleic acid encoding an ALAS; an additional copy of the P. pastoris ALAS nucleic acid (c.g., introduced recombinantly into P. pastoris) is considered to be exogenous. Similarly, an “exogenous” protein is a protein encoded by an exogenous nucleic acid.

In some instances, an exogenous nucleic acid can be a heterologous nucleic acid. As used herein, a “heterologous” nucleic acid refers to any nucleic acid sequence that is not native to an organism (e.g., a heterologous nucleic acid can be a nucleic acid from one microorganism (e.g., one genus or species of methylotrophic yeast, whether or not it has been codon-optimized) that is introduced into a different genus or species of methylotrophic yeast)). Similarly, a “heterologous” protein is a protein encoded by a heterologous nucleic acid.

A nucleic acid molecule is considered to be exogenous to a host organism when any portion thereof (e.g., a promoter sequence or a sequence of an encoded protein) is exogenous to the host organism. A nucleic acid molecule is considered to be heterologous to a host organism when any portion thereof (e.g., a promoter sequence or a sequence of an encoded protein) is heterologous to the host organism.

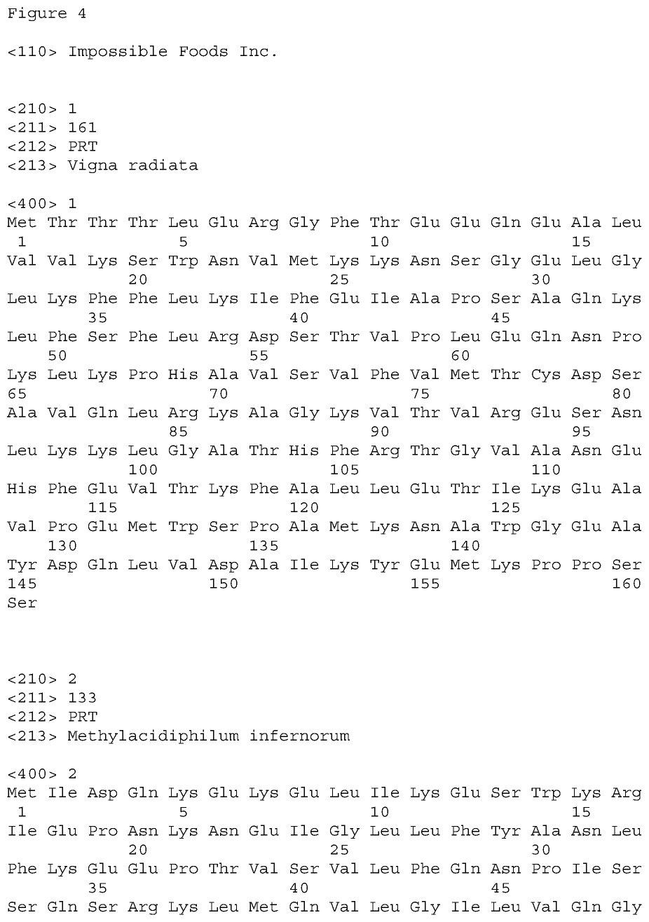

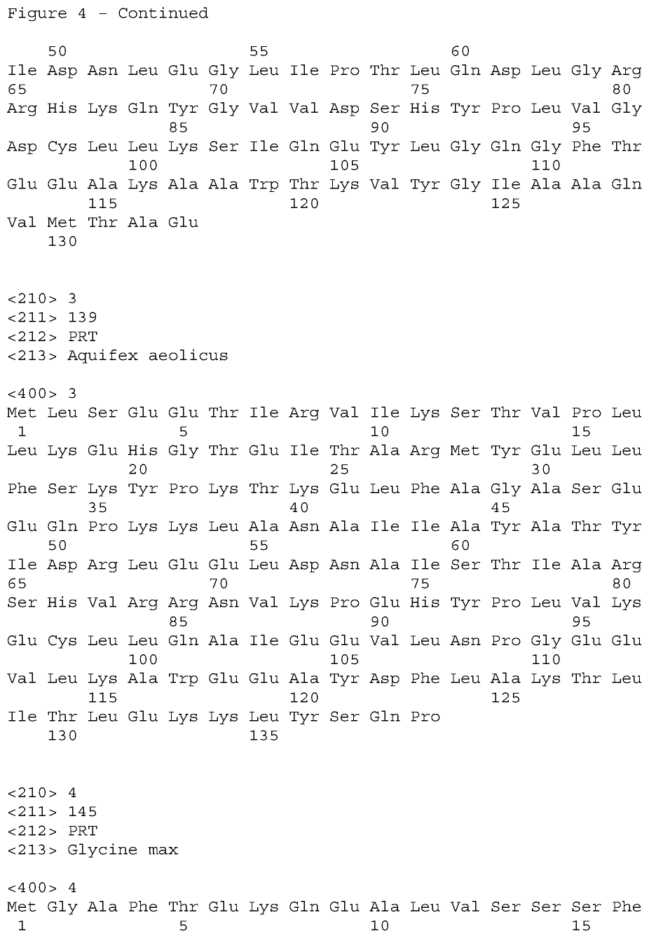

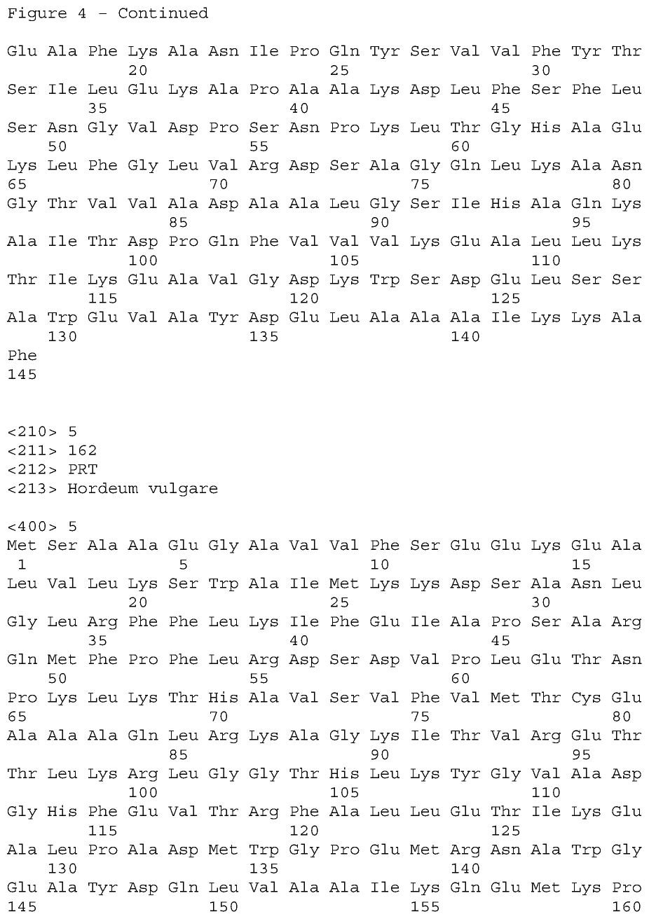

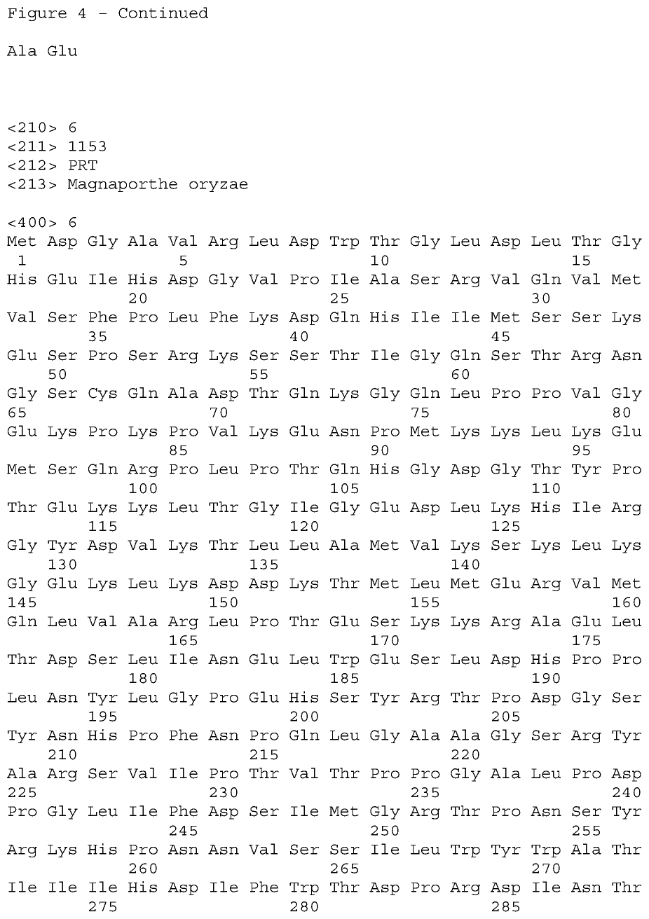

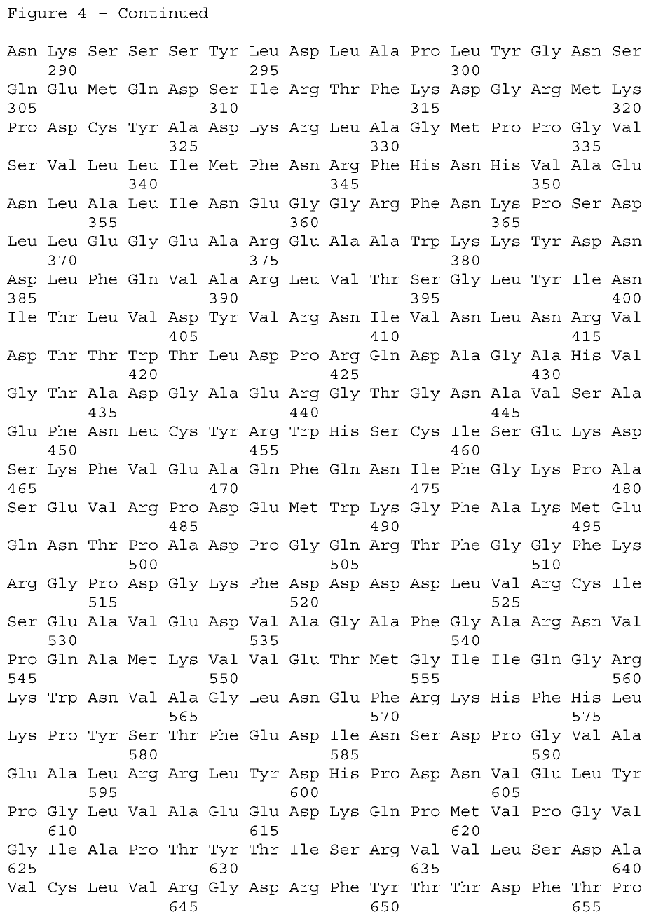

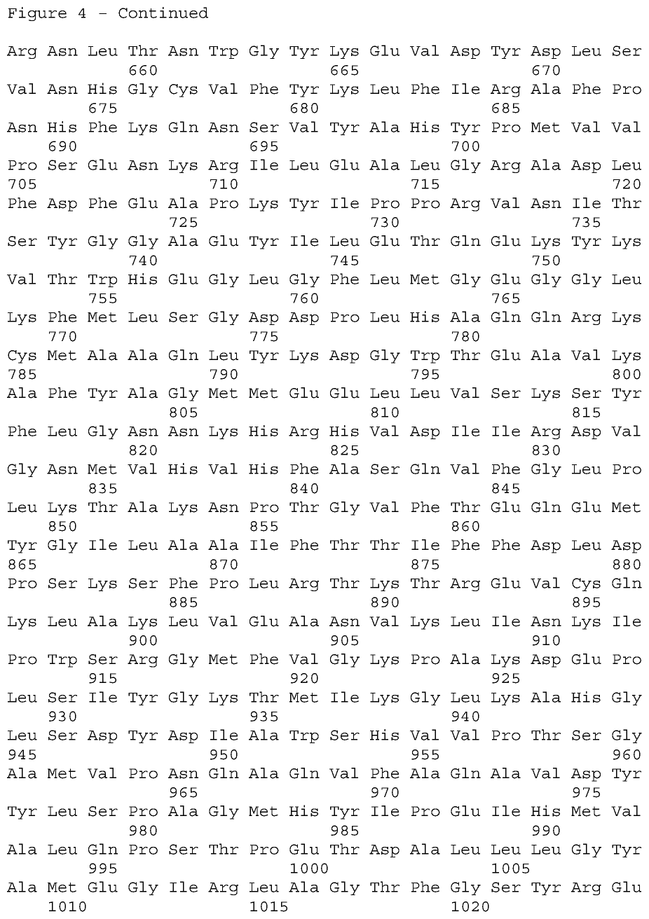

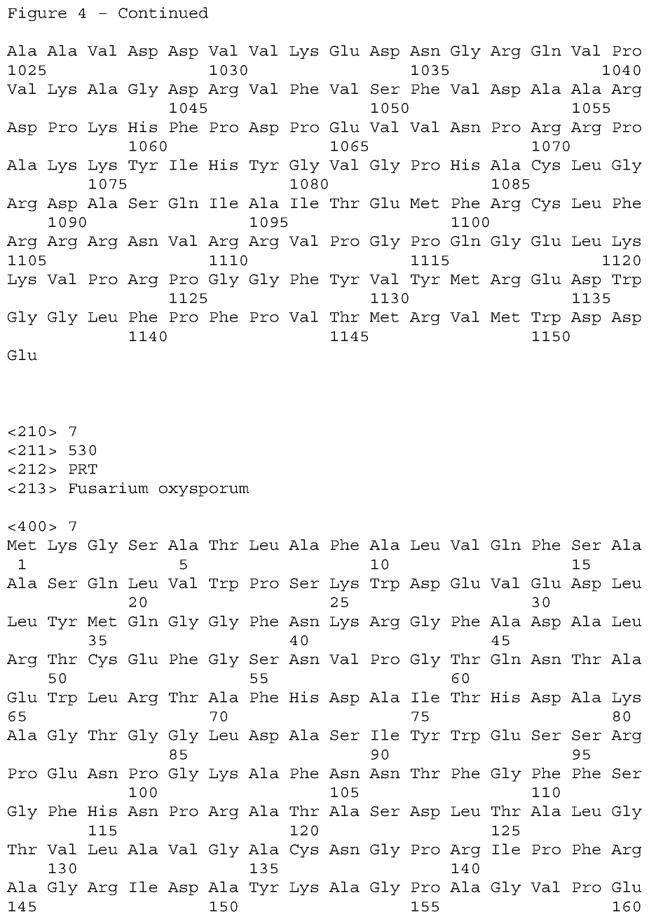

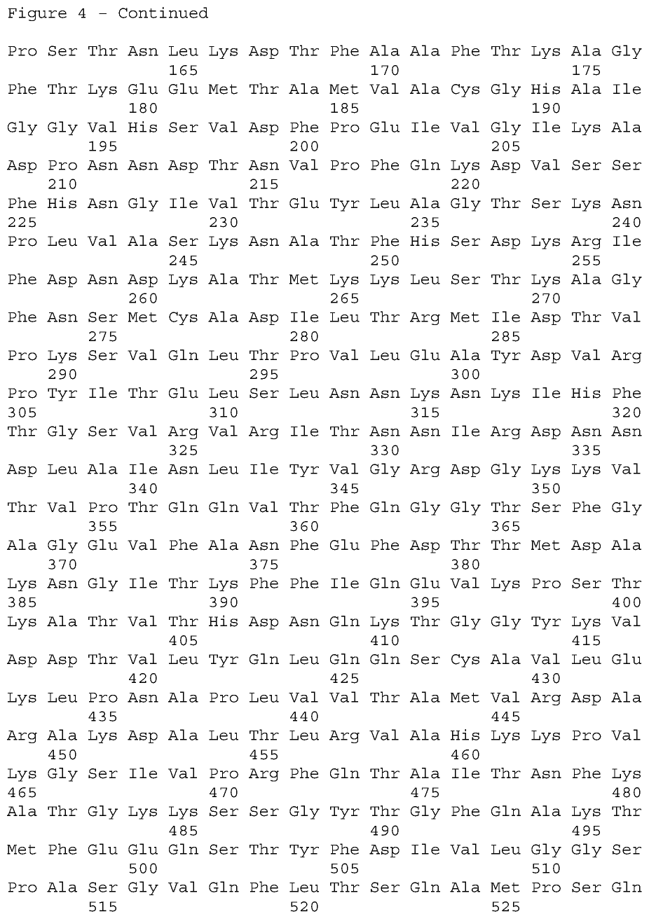

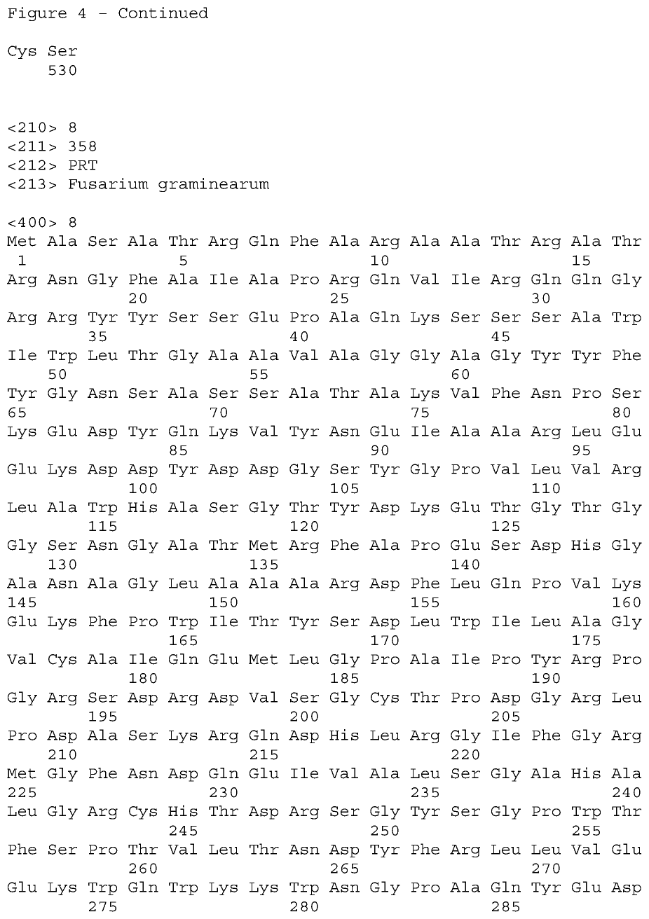

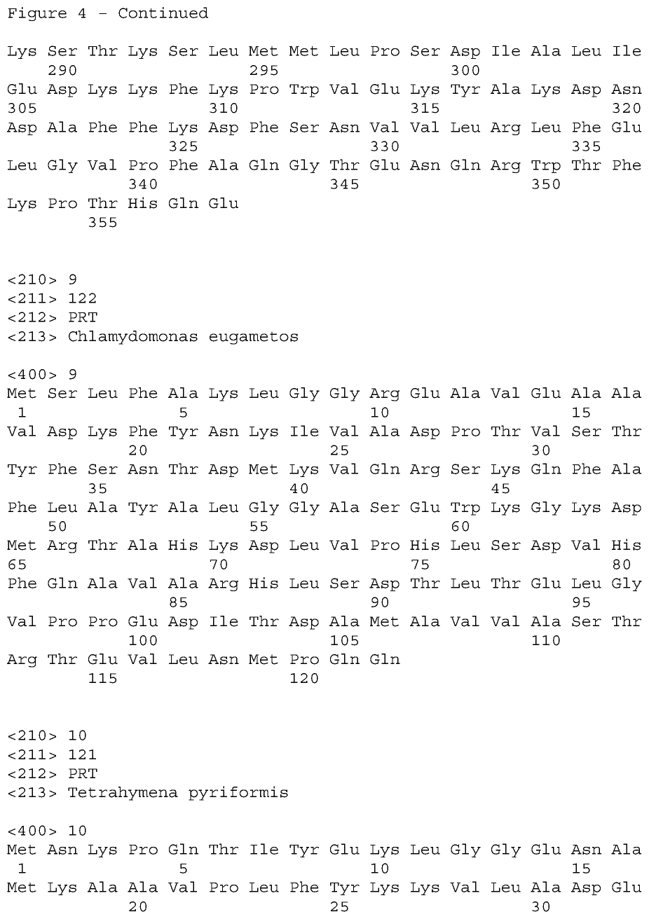

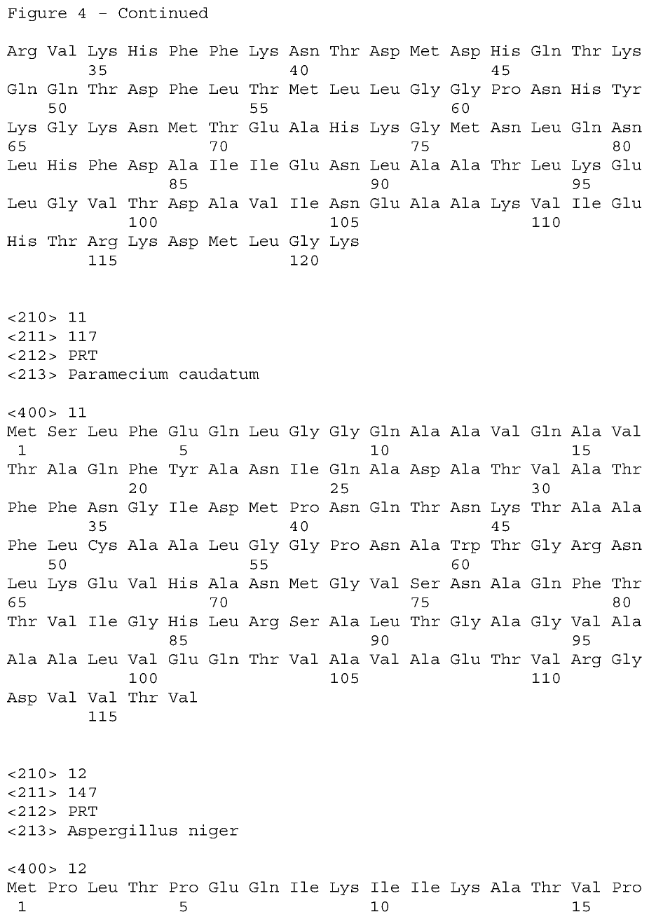

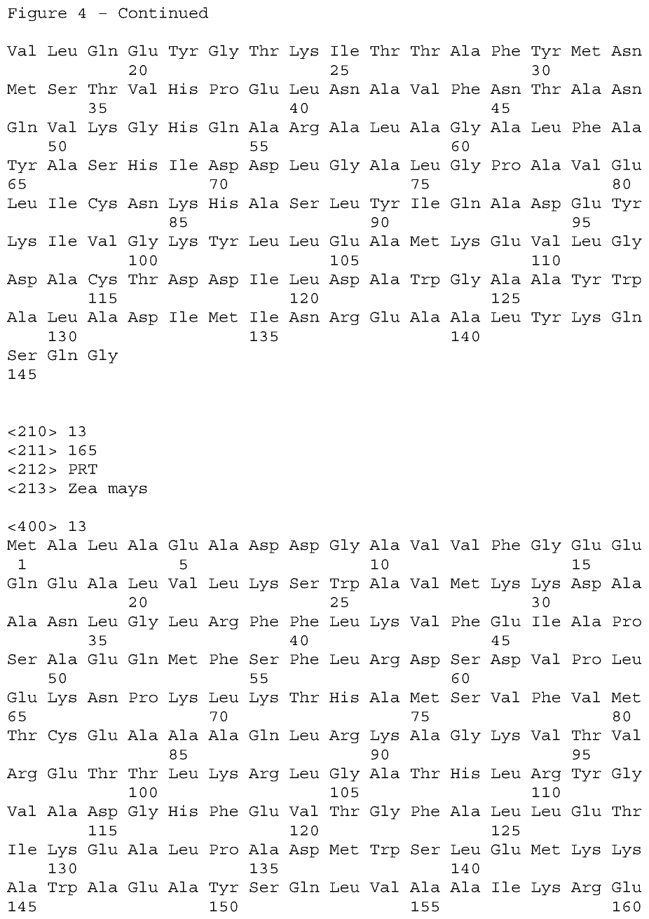

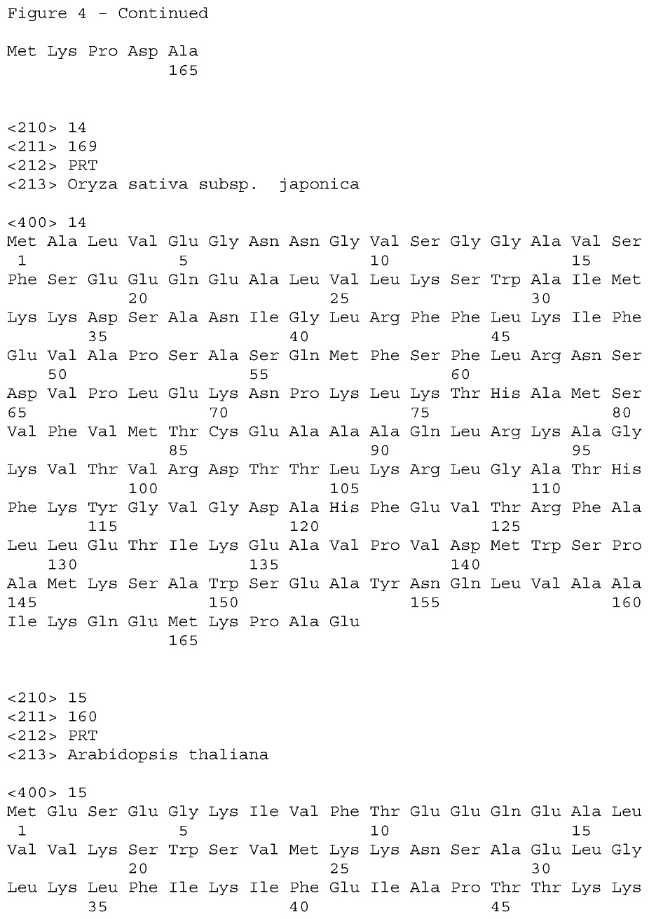

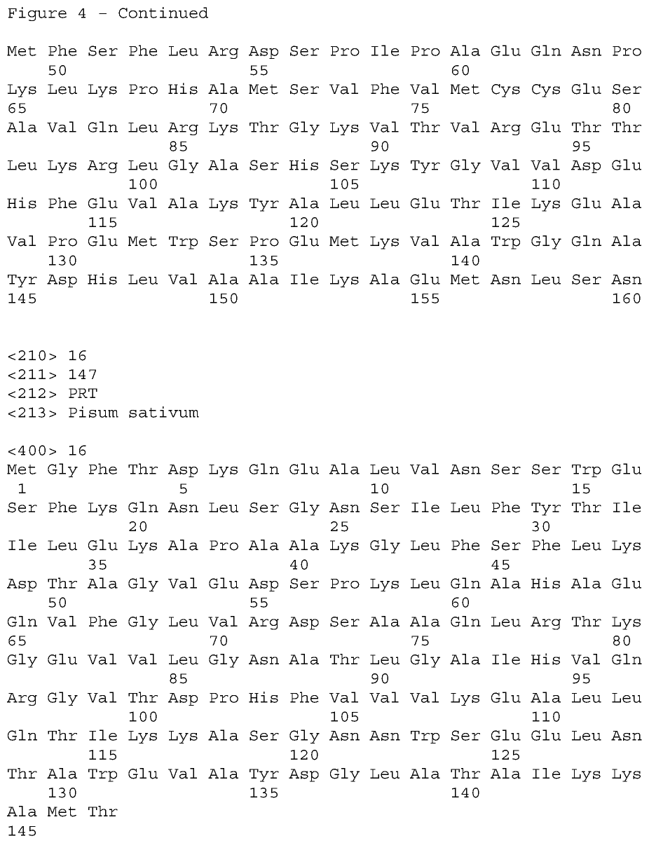

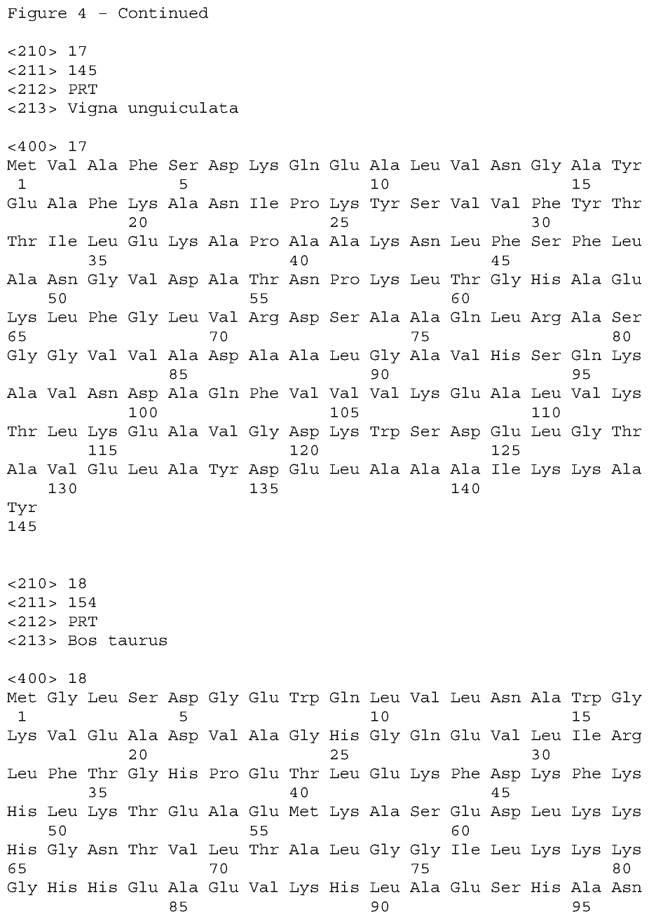

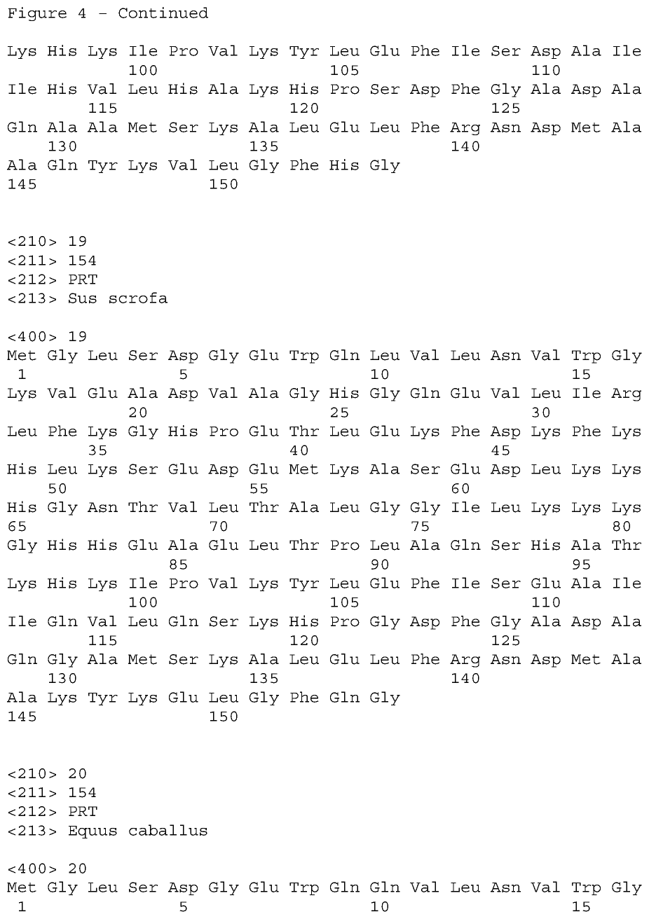

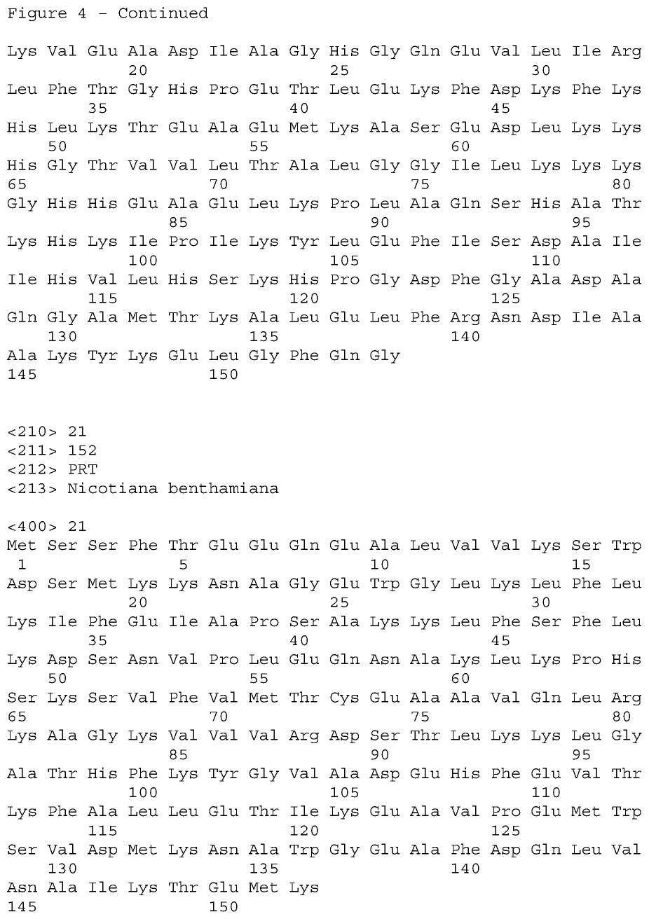

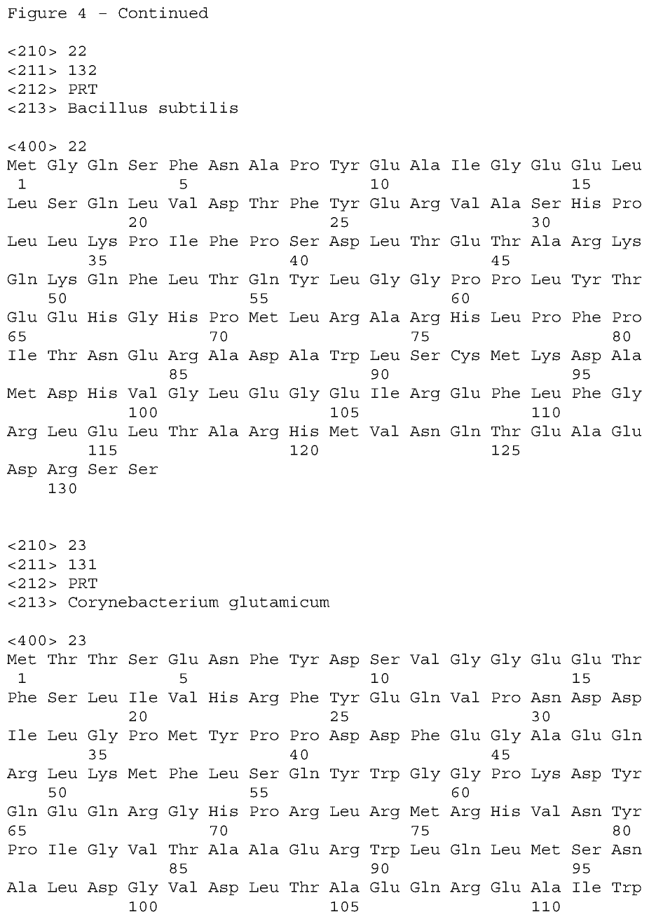

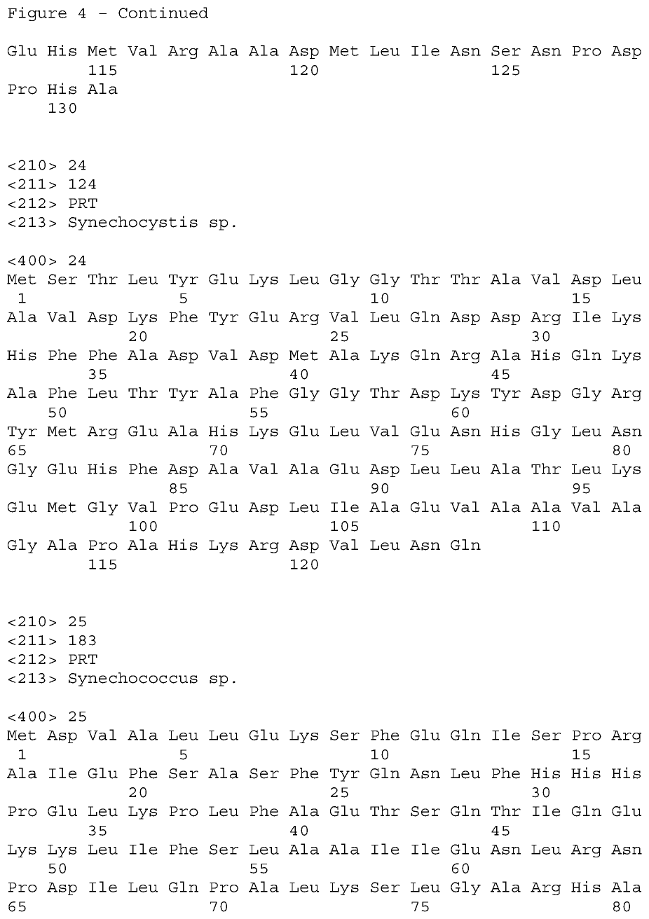

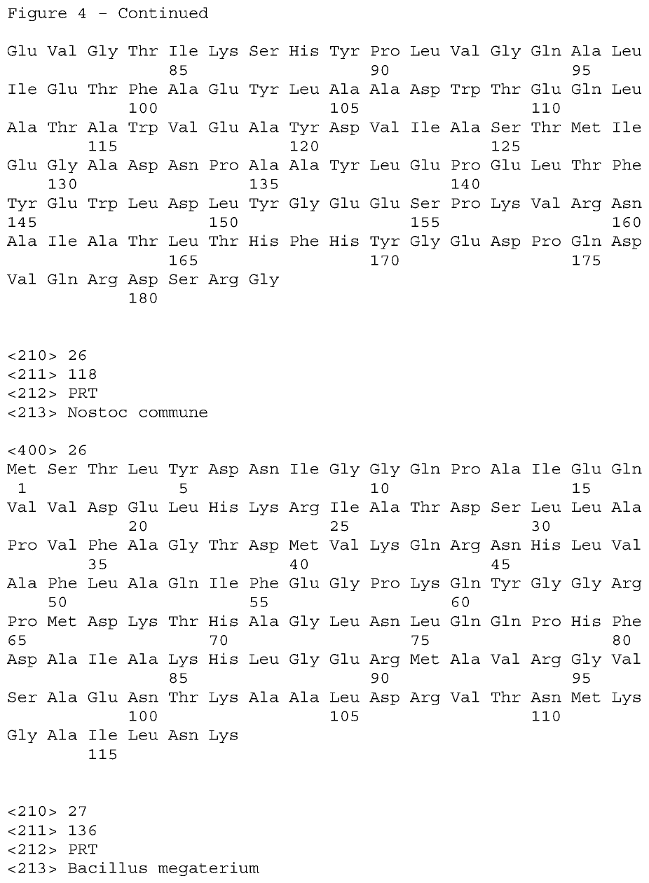

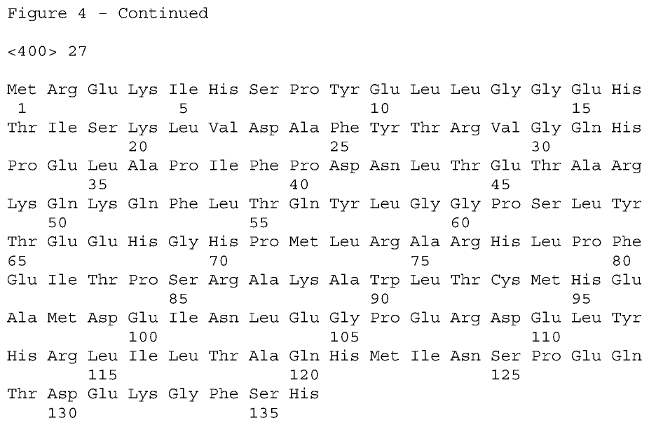

In some embodiments, this document provides a cell (e.g., fungal cell, such as an Aspergillus cell, a Trichoderma cell, or a yeast cell (e.g., a methylotrophic yeast cell)) comprising a first nucleic acid construct as disclosed herein and a second nucleic acid construct comprising a nucleotide sequence encoding a heme-binding protein. In some embodiments, the second nucleic acid encodes a heme-binding protein operably linked to the first promoter element. In some embodiments, the second nucleic acid encodes a heme-binding protein operably linked to a second promoter element. In some embodiments, a heme-binding protein can be an exogenous heme-binding protein. In some embodiments, a heme-binding protein can be a heterologous heme-binding protein. In some embodiments, a heme-binding protein can be selected from the group consisting of a globin (PF00042 in the Pfam database), a cytochrome (e.g., a cytochrome P450, a cytochrome a, a cytochrome b, a cytochrome c), a cytochrome c oxidase, a ligninase, a catalase, and a peroxidase. In some embodiments, a globin can be selected from the group consisting of an androglobin, a chlorocruorin, a cytoglobin, an erythrocruorin, a flavohemoglobin, a globin E, a globin X, a globin Y, a hemoglobin (e.g., a beta hemoglobin, an alpha hemoglobin), a histoglobin, a leghemoglobin, a myoglobin, a neuroglobin, a non-symbiotic hemoglobin, a protoglobin, and a truncated hemoglobin (e.g., a HbN, a HbO, a Glb3, a cyanoglobin). In some embodiments, the heme-binding protein can be a non-symbiotic hemoglobin. In some embodiments, the heme-binding protein can be a leghemoglobin. In some embodiments, the heme-binding protein can be soybean leghemoglobin (LegH). A reference amino acid sequence for LegH is provided in FIG. 4 as SEQ ID NO: 4. LegH is a protein that binds to heme, which results in a characteristic absorption at 415 nm and a distinct red color. The LegH protein (also known as LGB2) is naturally found in root nodules of soybean (see, for example, UniprotKB Accession No. P02236). Sec, also, WO 2014/110539 and WO 2014/110532, each of which is herein incorporated by reference in its entirety. In some embodiments, a heme-binding protein can have an amino acid sequence that is at least 70% (c.g., at least 75%, 80%, 85%, 90%, 91%, 92%, 93%, 94%, 95%, 96%, 97%, 98%, or 99%) identical to the amino acid sequence set forth in any of SEQ ID NOs: 1-27 (FIG. 4). In some embodiments, a heme-binding protein can have an amino acid sequence set forth in any of SEQ ID NOs: 1-27. In some embodiments, a heme-binding protein uses heme as a cofactor. As used herein, a “cofactor” is a molecule or ion that is directly involved in enzyme catalysis. In some embodiments, a heme-binding protein may or may not be an enzyme. In some embodiments, the heme-binding protein may or may not be part of the heme biosynthesis pathway. In some embodiments, the heme-binding protein may or may not be an ALAS. In some embodiments, the heme-binding protein may or may not be a ferrochelatase. In some embodiments, the heme-binding protein may or may not be a coprotoporphyrinogen oxidase. In some embodiments, the heme binding protein may or may not be ALAD, PBGD, UPG3S, UPG3D, COPROX, PROTOX, or FC. In some embodiments, a heme-binding protein has an amino acid sequence that is at least 70% (e.g., at least 75%, 80%, 85%, 90%, or 95%) identical to an amino acid sequence in any of SEQ ID NOs: 1-27. In some embodiments, a heme-binding protein is a bacteria-derived heme-binding protein, a yeast-derived heme-binding protein, an algae-derived heme-binding protein, a fungus-derived heme-binding protein, or a plant-derived heme-binding protein.

As used herein, a “bacteria-derived protein”, “yeast-derived protein”, “algae-derived protein”, “fungus-derived protein”, or “plant-derived protein” refers to the immediate source of the protein, and can mean any protein that is produced in a bacterium, a yeast, an algac, a fungus, or a plant, respectively, independently of whether the protein is natively expressed in the bacterium, yeast, algae, fungus, or plant, respectively.

Provided herein are methods of producing an ALAS protein. Also provided herein are methods of producing an ALAS protein using any of the cells (e.g., fungal cells, such as Aspergillus cells, Trichoderma cells, or yeast cells (e.g., methylotrophic yeast cells)) described herein. Also provided herein are methods of producing an ALAS protein using any of the nucleic acid constructs described herein. In some embodiments, provided herein are methods comprising expressing a first nucleic acid construct comprising a nucleotide sequence encoding an ALAS protein comprising a mutation in a first HRM. In some embodiments, the titer of an ALAS protein can be increased by at least 5% (e.g., at least 6%, 7%, 8%, 9%, 10%, 15%, 20%, 25%, 30%, 40%, 50%, 60%, 70%, 80%, 90%, 100%, 120%, 140%, 150%, 160%, 180%, 200%, 220%, 240%, 250%, or more) compared to a corresponding method lacking a nucleic acid construct comprising a nucleotide sequence encoding an ALAS protein comprising a mutation in a first HRM. In some embodiments, the titer of an ALAS protein can be increased by at least 5% (e.g., at least 6%, 7%, 8%, 9%, 10%, 15%, 20%, 25%, 30%, 40%, 50%, 60%, 70%, 80%, 90%, 100%, 120%, 140%, 150%, 160%, 180%, 200%, 220%, 240%, 250%, or more) compared to a corresponding method expressing a nucleic acid encoding an ALAS protein that does not comprise a mutation in a first HRM. In some embodiments, provided herein are methods comprising expressing a first nucleic acid construct comprising a nucleotide sequence encoding an ALAS protein comprising a mutation in a first HRM and a mutation in a second HRM. In some embodiments, the titer of an ALAS protein can be increased by at least 5% (e.g., at least 6%, 7%, 8%, 9%, 10%, 15%, 20%, 25%, 30%, 40%, 50%, 60%, 70%, 80%, 90%, 100%, 120%, 140%, 150%, 160%, 180%, 200%, 220%, 240%, 250%, or more) compared to a corresponding method lacking a nucleic acid construct comprising a nucleotide sequence encoding an ALAS protein comprising a mutation in a first HRM and a mutation in a second HRM. In some embodiments, the titer of an ALAS protein can be increased by at least 5% (e.g., at least 6%, 7%, 8%, 9%, 10%, 15%, 20%, 25%, 30%, 40%, 50%, 60%, 70%, 80%, 90%, 100%, 120%, 140%, 150%, 160%, 180%, 200%, 220%, 240%, 250%, or more) compared to a corresponding method expressing a nucleic acid encoding an ALAS protein that does not comprise a mutation in a first HRM and a mutation in a second HRM.

Provided herein are methods of producing a tetrapyrrole or a derivative thereof. Also provided herein are methods of producing a tetrapyrrole or a derivative thereof using any of the cells (e.g., fungal cells, such as Aspergillus cells, Trichoderma cells, or yeast cells (e.g., methylotrophic yeast cells))described herein. Also provided herein are methods of producing a tetrapyrrole or a derivative thereof using any of the nucleic acid constructs described herein. In some embodiments, provided herein are methods of producing a tetrapyrrole or a derivative thereof comprising expressing a first nucleic acid construct comprising a nucleotide sequence encoding an ALAS protein comprising a mutation in a first HRM. In some embodiments, the titer of a tetrapyrrole or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM. In some embodiments, the titer of a tetrapyrrole or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM. In some embodiments, provided herein are methods of producing a tetrapyrrole or a derivative thereof comprising expressing a first nucleic acid construct comprising a nucleotide sequence encoding an ALAS protein comprising a mutation in a first HRM and a second mutation in a second HRM. In some embodiments, the titer of a tetrapyrrole or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM and a mutation in a second HRM. In some embodiments, the titer of a tetrapyrrole or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM and a mutation in a second HRM. In some embodiments, a tetrapyrrole or a derivative thereof is a chlorin (e.g., a chlorophyll) or a derivative thereof. In some embodiments, a tetrapyrrole or a derivative thereof is a precorrin, a co-precorrin, a corrin (e.g., vitamin B12), or a derivative thereof. In some embodiments, a tetrapyrrole or a derivative thereof is a porphyrin or a derivative thereof. In some embodiments, a tetrapyrrole or a derivative thereof is a heme or a derivative thereof. In some embodiments, a tetrapyrrole or a derivative thereof is heme B. In some embodiments, a tetrapyrrole or a derivative thereof is a metabolic product of heme (e.g., bilirubin or a derivative thereof).

Provided herein are methods of producing an intermediate of the heme biosynthesis pathway, a heme (e.g., heme B, heme o, heme a, heme c), a corrin (e.g., Vitamin B12), a chlorophyll, or a derivative thereof. Also provided herein are methods of producing an intermediate of the heme biosynthesis pathway, a heme, a corrin, a chlorophyll, or a derivative thereof using any of the cells (e.g., fungal cells, such as Aspergillus cells, Trichoderma cells, or yeast cells (e.g., methylotrophic yeast cells))described herein. Also provided herein are methods of producing an intermediate of the heme biosynthesis pathway, a heme, a corrin, a chlorophyll, or a derivative thereof using any of the nucleic acid constructs described herein. In some embodiments, provided herein are methods of producing an intermediate of the heme biosynthesis pathway, a heme, a corrin, a chlorophyll, or a derivative thereof comprising expressing a first nucleic acid construct comprising a nucleotide sequence encoding an ALAS protein comprising a mutation in a first HRM. In some embodiments, the titer of an intermediate of the heme biosynthesis pathway, a heme, a corrin, a chlorophyll, or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM. In some embodiments, the titer of an intermediate of the heme biosynthesis pathway, a heme, a corrin, a chlorophyll, or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM. In some embodiments, provided herein are methods of producing an intermediate of the heme biosynthesis pathway, a heme, a corrin, a chlorophyll, or a derivative thereof comprising expressing a first nucleic acid construct comprising a nucleotide sequence encoding an ALAS protein comprising a mutation in a first HRM and a mutation in a second HRM. In some embodiments, the titer of an intermediate of the heme biosynthesis pathway, a heme, a corrin, a chlorophyll, or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM and a mutation in a second HRM. In some embodiments, the titer of an intermediate of the heme biosynthesis pathway, a heme, a corrin, a chlorophyll, or a derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM and a mutation in a second HRM. In some embodiments, an intermediate of the heme biosynthesis pathway, a heme, or a derivative thereof is a heme. In some embodiments, an intermediate of the heme biosynthesis pathway, a heme, or a derivative thereof is heme B.

Provided herein are methods of producing a heme (e.g., heme B, heme o, heme a, heme c) or a derivative thereof. Also provided herein are methods of producing a heme or derivative thereof using any of the cells (e.g., fungal cells, such as Aspergillus cells, Trichoderma cells, or yeast cells (e.g., methylotrophic yeast cells)) described herein. Also provided herein are methods of producing a heme or derivative thereof using any of the nucleic acid constructs described herein. In some embodiments, provided herein are methods of producing a heme or derivative thereof comprising expressing a first nucleic acid construct comprising a nucleotide sequence encoding an ALAS protein comprising a mutation in a first HRM. In some embodiments, the titer of a heme or derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM. In some embodiments, the titer of a heme or derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM. In some embodiments, provided herein are methods of producing a heme or derivative thereof comprising expressing a first nucleic acid construct comprising a nucleotide sequence encoding an ALAS protein comprising a mutation in a first HRM and a mutation in a second HRM. In some embodiments, the titer of a heme or derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method lacking a nucleic acid construct comprising a nucleotide sequence encoding an ALAS protein comprising a mutation in a first HRM and a mutation in a second HRM. In some embodiments, the titer of a heme or derivative thereof can be increased by at least 5% (e.g., at least 10%, 20%, 30%, 40%, 50%, 60%, 70%, 80%, 90%, 100%, 120%, 140%, 150%, 160%, 180%, 200%, 220%, 240%, 250%, 280%, 300%, 320%, 340%, 350%, or more) compared to a corresponding method expressing a nucleic acid encoding an ALAS protein that does not comprise a mutation in a first HRM and a mutation in a second HRM. In some embodiments, a heme or a derivative thereof is heme B.

Provided herein are methods of producing a heme-binding protein. Also provided herein are methods of producing a heme-binding protein using any of the cells (e.g., fungal cells, such as Aspergillus cells, Trichoderma cells, or yeast cells (e.g., methylotrophic yeast cells))described herein. Also provided herein are methods of producing a heme-binding protein using any of the nucleic acid constructs described herein. In some embodiments, provided herein are methods comprising expressing a first nucleic acid construct comprising a nucleotide sequence encoding an ALAS protein comprising a mutation in a first HRM and expressing a second nucleic acid construct comprising a nucleotide sequence encoding a heme-binding protein. In some embodiments, provided herein are methods comprising expressing a first nucleic acid construct comprising a nucleotide sequence encoding an ALAS protein comprising a mutation in a first HRM and expressing a second nucleic acid construct comprising a nucleotide sequence encoding a heme-binding protein. In some embodiments, provided herein are methods comprising expressing a first nucleic acid construct comprising a nucleotide sequence encoding an ALAS protein comprising a mutation in a first HRM and a mutation in a second HRM and expressing a second nucleic acid construct comprising a nucleotide sequence encoding a heme-binding protein. In some embodiments, provided herein are methods comprising expressing a first nucleic acid construct comprising a nucleotide sequence encoding an ALAS protein comprising a mutation in a first HRM and a mutation in a second HRM and expressing a second nucleic acid construct comprising a nucleotide sequence encoding a heme-binding protein. In some embodiments of any of the methods described herein, the methods allow for an increase in the titer of a heme-binding protein. In some embodiments, the titer of a heme-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method lacking a nucleic acid construct comprising a nucleotide sequence encoding an ALAS protein comprising a mutation in a first HRM. In some embodiments, the titer of a heme-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method expressing a nucleic acid encoding an ALAS protein that does not comprise a mutation in a first HRM. In some embodiments, the titer of a heme-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method lacking a nucleic acid construct comprising a nucleotide sequence encoding an ALAS protein comprising a mutation in a first HRM and a mutation in a second HRM. In some embodiments, the titer of a heme-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method expressing a nucleic acid encoding an ALAS protein that does not comprise a mutation in a first HRM and a mutation in a second HRM.

Provided herein are methods of producing a tetrapyrrole-binding protein. Also provided herein are methods of producing a tetrapyrrole-binding protein using any of the cells (e.g., fungal cells, such as Aspergillus cells, Trichoderma cells, or yeast cells (e.g., methylotrophic yeast cells)) described herein. Also provided herein are methods of producing a tetrapyrrole-binding protein using any of the nucleic acid constructs described herein. In some embodiments, provided herein are methods comprising expressing a first nucleic acid construct comprising a nucleotide sequence encoding an ALAS protein comprising a mutation in a first HRM and expressing a second nucleic acid construct comprising a nucleotide sequence encoding a tetrapyrrole-binding protein. In some embodiments, provided herein are methods comprising expressing a first nucleic acid construct comprising a nucleotide sequence encoding an ALAS protein comprising a mutation in a first HRM and expressing a second nucleic acid construct comprising a nucleotide sequence encoding a tetrapyrrole-binding protein. In some embodiments, provided herein are methods comprising expressing a first nucleic acid construct comprising a nucleotide sequence encoding an ALAS protein comprising a mutation in a first HRM and a mutation in a second HRM and expressing a second nucleic acid construct comprising a nucleotide sequence encoding a tetrapyrrole-binding protein. In some embodiments, provided herein are methods comprising expressing a first nucleic acid construct comprising a nucleotide sequence encoding an ALAS protein comprising a mutation in a first HRM and a mutation in a second HRM and expressing a second nucleic acid construct comprising a nucleotide sequence encoding a tetrapyrrole-binding protein. In some embodiments of any of the methods described herein, the methods allow for an increase in the titer of a tetrapyrrole-binding protein. In some embodiments, the titer of a tetrapyrrole-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method lacking a nucleic acid construct comprising a nucleotide sequence encoding an ALAS protein comprising a mutation in a first HRM. In some embodiments, the titer of a tetrapyrrole-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method expressing a nucleic acid encoding an ALAS protein that does not comprise a mutation in a first HRM. In some embodiments, the titer of a tetrapyrrole-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method lacking a nucleic acid construct comprising a nucleotide sequence encoding an ALAS protein comprising a mutation in a first HRM and a mutation in a second HRM. In some embodiments, the titer of a tetrapyrrole-binding protein can be increased by at least 5% (e.g., at least 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, or more) compared to a corresponding method expressing a nucleic acid encoding an ALAS protein that does not comprise a mutation in a first HRM and a mutation in a second HRM. In some embodiments, a tetrapyrrole is a chlorin (e.g., a chlorophyll) or a derivative thereof. In some embodiments, a tetrapyrrole is a precorrin, a co-precorrin, a corrin (e.g., vitamin B12), or a derivative thereof. In some embodiments, a tetrapyrrole is a porphyrin or a derivative thereof. In some embodiments, a tetrapyrrole is a heme or a derivative thereof. In some embodiments, a tetrapyrrole is heme B. In some embodiments, a tetrapyrrole is a metabolic product of heme (e.g., bilirubin or a derivative thereof).

As used herein, a “corresponding method” is a method that is essentially identical to a reference method in all ways except for the identified difference. For example, a corresponding method expressing a nucleic acid encoding an ALAS protein that does not comprise a mutation in a first HRM and a mutation in a second HRM would be the same in all aspects (e.g., genetic makeup of cell, temperature and time of culture, and so forth), except that the corresponding method would express a nucleic acid encoding an ALAS protein that does not comprise a mutation in a first HRM and a mutation in a second HRM.

Genetically engineering a cell (e.g., fungal cell, such as an Aspergillus cell, a Trichoderma cell, or a yeast cell (e.g., a methylotrophic yeast cell)) typically includes introducing a recombinant nucleic acid molecule (also called a nucleic acid construct) into the cell. As described herein, a recombinant nucleic acid molecule typically includes an exogenous nucleic acid that encodes a protein (e.g., a protein involved in heme biosynthesis, a heme-binding protein, or a transcription factor) operably linked to at least one promoter element (c.g., an inducible or constitutive promoter element). In some embodiments, a recombinant nucleic acid molecule can include a linear sequence of two or more protein-coding sequences operably linked to the same or separate promoter elements (e.g., a first promoter operably linked to a first nucleic acid encoding a first protein and a second promoter operably linked to a second nucleic acid encoding a second protein, or a promoter operably linked to a first nucleic acid encoding a first protein and a second nucleic acid encoding a second protein). In some cases, a recombinant nucleic acid molecule including at least one promoter operably linked to a nucleotide sequence encoding a protein can be called a cassette.

A recombinant nucleic acid can include expression elements. Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins.

Nucleic acids can be detected using any number of amplification techniques (sec, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188) with an appropriate pair of oligonucleotides (e.g., primers). A number of modifications to the original PCR method have been developed and can be used to detect selected nucleic acids.

Methanol utilization is typically initiated by the conversion of methanol to formaldehyde by the action of alcohol oxidase. Pichia pastoris contains two genes for alcohol oxidases, AOX1 and AOX2. Strains with reduced alcohol oxidase activity (“methanol utilization slow” or MutS strains) can produce more of a recombinant protein expressed from the AOX1 promoter than strains that do not have reduced alcohol oxidase activity. Strains mutated in both AOX genes and completely lacking alcohol oxidase activity cannot metabolize methanol, but can still be induced for expression from the AOX1 promoter by methanol. These strains retain the ability to use other carbon sources for growth, but still express heterologous proteins from the AOX1 promoter upon the addition of methanol. Because these strains do not metabolize methanol (“methanol utilization minus” or Mut- strains), much less methanol is required for induction of protein expression, and strains carrying these mutations avoid issues related to methanol feeding in large-scale fermentations. See, for example, Chiruvolu et al., 1997, Enzyme Microb. Technol., 21:277-83.

Suitable transcription factors, and nucleic acids encoding transcription factors (e.g., exogenous nucleic acids encoding transcription factors), include, for example, Mxr1 from a P. pastoris. A representative K. pastoris Mxr1 nucleic acid sequence can be found, for example, in GenBank Accession No. DQ395124, while a representative K. pastoris Mxr1 protein sequence can be found, for example, in GenBank Accession No. ABD57365. In some embodiments, the transcription factor is a Mit1 sequence from K. phaffii (see, for example, UniParc Accession No. UPI0001A4D18B). Suitable transcription factors also can be found in Hansemila polymorpha (e.g., the Adr1 sequence; see, for example, GenBank Accession No. AEOI02000005, bases 858873 to 862352, for the nucleic acid sequence and GenBank Accession No. ESX01253 for the amino acid sequence) and Candida boidinii (e.g., the Trm1 sequence; see, for example, GenBank Accession No. AB365355 for the nucleic acid sequence and GenBank Accession No. BAF99700 for the amino acid sequence; and Trm2 sequence; see, for example, GenBank Accession No. AB548760 for the nucleic acid sequence and GenBank Accession No. BAJ07608 for the amino acid sequence).

Transcription factors such as Mxr1 may be normally expressed at low levels. In some embodiments, it is desirable to place an exogenous nucleic acid (e.g., a transcription factor) under control of a promoter that is inducible.

Methanol-regulated transcription factors in Pichia can bind to an AOX1 promoter and act cooperatively with Mxr1 to activate transcription from an AOX1 promoter. In some embodiments, two methanol-regulated transcription factors (e.g., Mxr1 and Mit1) can be operably linked to a methanol inducible promoter element.

There are a number of inducible promoters that can be used when genetically engineering cells (e.g., fungal cells such as Aspergillus, Trichoderma cells, or yeast cells (e.g., methylotrophic yeast cells)). Suitable methanol inducible promoters include pAOX1, (e.g., a pAOX1 such as the promoter for K. pastoris AOX1 (see, for example, the promoter for GenBank Accession No. U96967.1) or a promoter described in U.S. Provisional Patent Application No. 62/835,338, filed Apr. 17, 2019, incorporated herein by reference in its entirety), as well as other methanol-inducible promoters, or promoter elements therefrom. These include, without limitation, a pAOX2 promoter (e.g., from K. phaffii or K. pastoris (see, for example, GenBank Accession No. X79871.1)), an alcohol oxidase (AOD1) promoter from, e.g., Candida boidinii (see, for example, GenBank Accession No. E06147.1), the alcohol oxidase (MOX) promoter from Hansenula polymorpha (see, for example, GenBank Accession No. AJ313360.1), the MOD1 or MOD2 promoter from Pichia methanolica (see, for example, Raymond et al., 1998, Yeast, 14:11-23; and Nakagawa et al., 1999, Yeast, 15:1223-30), the DHAS promoter from P. pastoris (see, for example, the promoter for GenBank Accession No. FJ752551) or a promoter element therefrom, the formaldehyde dehydrogenase (FLD1) promoter from K. pastoris (see, for example, GenBank Accession No. KJ755994.1), or the PEX8 promoter from P. pastoris (see, for example, Kranthi et al., 2010, Yeast, 27:705-11). Typically, these promoters can be induced by methanol. Suitable constitutive promoters and constitutive promoter elements include, without limitation, the P. pastoris promoter (or a portion thereof) from the transcriptional elongation factor EF-1α gene (TEF1), which is strongly transcribed in a constitutive manner. Other suitable constitutive promoters (or promoter elements therefrom) also can be used, including, without limitation, the glyceraldehyde-3-phosphate dehydrogenase (GAPDH) promoter from K. pastoris (see, for example, the promoter for GenBank Accession No. U62648.1), the promoter from the potential glycosyl phosphatidyl inositol (GPI)-anchored protein, GCW14p (PAS_chr1-4_0586) from K. phaffii (see, for example, the promoter for GenBank Accession No. XM_002490678), and the promoter from the 3-phosphoglycerate kinase gene (PGK1) from K. pastoris (see, for example, GenBank Accession No. AY288296). It will be appreciated that a choice of promoter may be influenced by the expression system. For example, for expression in K. phaffii, a K. phaffii promoter might be chosen, while for expression in C. boidinii, a C. boidinii promoter might be chosen. However, in some cases, a promoter from one organism (e.g., K. phaffii) may be appropriate to be used in another organism (e.g., C. boidinii or K. pastoris). Further, it is noted that a combination of inducible (e.g., methanol-inducible) and constitutive promoters (or promoter elements therefrom) can be combined to further increase the expression of any of the nucleic acids operably linked thereto.

Any of the encoded proteins as described herein can be operably linked to an inducible promoter element (e.g., a methanol-inducible promoter element) or a constitutive promoter element. Inducible promoters and elements therefrom are discussed above. Constitutive promoters and constitutive promoter elements are known in the art. For example, a commonly used constitutive promoter from P. pastoris is the promoter, or a portion thereof, from the transcriptional elongation factor EF-1α gene (TEF1), which is strongly transcribed in a constitutive manner. Other constitutive promoters, or promoter elements therefrom, however, can be used, including, without limitation, the glyceraldehyde-3-phosphate dehydrogenase (GAPDH) promoter from K. pastoris (see, for example, the promoter for GenBank Accession No. U62648.1), the promoter from the potential glycosyl phosphatidyl inositol (GPI)-anchored protein, GCW14p (PAS_chr1-4_0586), from K. phaffii (see, for example, the promoter for GenBank Accession No. XM_002490678), or the promoter from the 3-phosphoglycerate kinase gene (PGK1) from K. pastoris (see, for example, the promoter for GenBank Accession No. AY288296).

In some embodiments, any of the cells (e.g., fungal cell, such as an Aspergillus cell, a Trichoderma cell, or a yeast cell (e.g., a methylotrophic yeast cell)) herein can further include a third nucleic acid construct comprising a nucleotide sequence encoding a third protein operably linked to the first promoter element, the second promoter element, or a third promoter element. In some embodiments, any of the cells (e.g., fungal cell, such as an Aspergillus cell, a Trichoderma cell, or a yeast cell (e.g., a methylotrophic yeast cell)) herein can further include a fourth nucleic acid construct comprising a nucleotide sequence encoding a fourth protein operably linked to the first promoter element, the second promoter element, the third promoter element, or a fourth promoter element. In some embodiments, the third protein can be a transcription factor. In some embodiments, the fourth protein can be a transcription factor. In some embodiments, any of the promoter elements herein (e.g., a first promoter element, a second promoter element, a third promoter element, or a fourth promoter element) can contain one or more recognition sequences for a transcription factor. Therefore, in some embodiments, a feedback loop may be constructed such that the transcription factor drives the expression of additional copies of the transcription factor, as well as the expression of one or more of an ALAS and a heme-binding protein. In some embodiments, a transcription factor can be Mxr1 (see, e.g., U.S. Pat. No. 9,938,327, which is incorporated by reference in its entirety). In some embodiments, a third protein can be a protein involved in heme biosynthesis. In some embodiments, a fourth protein can be a protein involved in heme biosynthesis. In some embodiments, a protein involved in heme biosynthesis can be selected from the group consisting of δ-aminolevulinic acid dehydratase (ALAD), porphobilinogen deaminase (PBGD), uroporphyrinogen III synthase (UPG3S), uroporphyrinogen III decarboxylase (UPG3D), coprotoporphyrinogen oxidase (COPROX), protoporphyrinogen IX oxidase (PROTOX), and/or ferrochelatase (FC). In some embodiments, a protein involved in heme biosynthesis can be selected from the group consisting of δ-aminolevulinic acid dehydratase (ALAD), porphobilinogen deaminase (PBGD), uroporphyrinogen III synthase (UPG3S), uroporphyrinogen III decarboxylase (UPG3D), coprotoporphyrinogen oxidase (COPROX), and/or protoporphyrinogen IX oxidase (PROTOX).

Previous studies in Saccharomyces cerevisiae identified ALA dehydratase and porphobilinogen deaminase as rate limiting enzymes in heme biosynthesis (see, for example, Hoffman et al., 2003, Biochem. Biophys. Res. Commun., 310(4):1247-53). However, heterologous expression of individual heme enzymes in P. pastoris from the glyceraldehyde-3-phosphate dehydrogenase (GAP) promoter failed to overcome limitations associated with the expression of a recombinant protein containing a heme (see Krainer et al., 2015, Microb. Cell Fact., 13;14:4). Expression of a recombinant heme containing protein in P. pastoris can be achieved by co-expressing one or more enzymes of the heme biosynthetic pathway from methanol-inducible promoters, although it would be appreciated that one or more of the genes involved in the heme biosynthetic pathway could be expressed from one or more constitutive promoters (see, e.g., U.S. Pat. No. 9,938,327, which is incorporated by reference in its entirety).

In addition, it is noted that a first nucleic acid encoding a first protein (e.g., an ALAS protein) operably linked to a promoter element as described herein can be physically separate from a second nucleic acid encoding a second protein (e.g., a heme-binding protein) operably linked to a promoter element (that is, the first and second nucleic acids can be completely separate molecules). Alternatively, a first nucleic acid encoding a first protein operably linked to a promoter element and a second nucleic acid encoding a second protein operably linked to a promoter element can be included in the same nucleic acid construct. In some embodiments, a first nucleic acid encoding a first protein operably linked to a promoter element can be contiguous with a second nucleic acid encoding a second protein operably linked to a promoter element. It would be appreciated by a skilled artisan that, if the second nucleic acid molecule encoding a second protein is contiguous with the first nucleic acid encoding a protein of interest, a single promoter, or promoter element therefrom, can be used to drive transcription of both or all of the genes (e.g., the nucleic acid encoding the first protein as well as the second protein).

Methods of introducing nucleic acids into cells (e.g., fungal cells, such as Aspergillus cells, Trichoderma cells, or yeast cells (e.g., methylotrophic yeast cells))are known in the art, and include, without limitation, transduction, electroporation, biolistic particle delivery, and chemical transformation.

In addition, methods of culturing cells (e.g., fungal cells, such as Aspergillus cells, Trichoderma cells, or yeast cells (e.g., methylotrophic yeast cells))are known in the art. See, for example, Pichia Protocols, Methods In Molecular Biology, 389, Cregg, Ed., 2007, 2^{nd} Ed., Humana Press, Inc. Under some circumstances, it may be desirable to introduce or add methanol to the culture media, although, as demonstrated herein, methanol is not required to obtain efficient expression at high levels of one or more proteins of interest. Under some circumstances (e.g., when one or more nucleic acids encoding enzyme(s) involved in heme biosynthesis are expressed), it may be desirable to supplement the culture media with iron or a pharmaceutically or metabolically acceptable (or GRAS) salt thereof.

The recombinant nucleic acid molecules described herein can be stably integrated into the genome of the cell (e.g., the fungal cell, such as Aspergillus cell, Trichoderma cell, or yeast cell (e.g., the methylotrophic yeast cell)), or can be extrachromosomally expressed from a replication-competent plasmid. Methods of achieving both are known and used in the art.

The methods provided herein also can include purifying the expressed protein. As used herein, an “enriched” protein is a protein that accounts for at least 5% (e.g., at least 6%, 7%, 8%, 9%, 10%, 15%, 20%, 25%, 30%, 35%, 40%, 45%, 50%, 55%, 60%, or more) by dry weight, of the mass of the production cell (e.g., fungal cell, such as Aspergillus cell, Trichoderma cell, or yeast cell (e.g., methylotrophic yeast cell)), or at least 10% (e.g., at least 15%, 20%, 25%, 30%, 35%, 40%, 45%, 50%, 55%, 60%, 65%, 70%, 75%, 80%, 90%, 95%, or 99%) by dry weight, the mass of the production cell lysate (e.g., excluding cell wall or membrane material). As used herein, a “purified” protein is a protein that has been separated or purified from cellular components that naturally accompany it. Typically, the protein is considered “purified” when it is at least 60% (c.g., at least 65%, 70% 75%, 80%, 85%, 90%, 95%, or 99%) by dry weight, free from the proteins and naturally occurring molecules with which it is naturally associated.

As used herein, nucleic acids can include DNA and RNA, and includes nucleic acids that contain one or more nucleotide analogs or backbone modifications. A nucleic acid can be single stranded or double stranded, which usually depends upon its intended use. Also provided are nucleic acids and polypeptides that differ from a given sequence. Nucleic acids and polypeptides can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a given nucleic acid or polypeptide sequence.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13): 3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis, transposon mutagenesis, chemical mutagenesis, UV mutagenesis or radiation induced mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al., 1978, Atlas of Protein Sequence and Structure, 5(Suppl. 3):345-352, which provides frequency tables for amino acid substitutions), and a non-conservative substitution is one in which an amino acid residue is replaced with an amino acid residue that does not have a similar side chain. Nucleic acid and/or polypeptide sequences may be modified as described herein to improve one or more properties such as, without limitation, increased expression (e.g., transcription and/or translation), tighter regulation, deregulation, loss of catabolite repression, modified specificity, secretion, thermostability, solvent stability, oxidative stability, protease resistance, catalytic activity, and/or color.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

Vectors as described herein can be introduced into a host cell (e.g., fungal cell, such as an Aspergillus cell, a Trichoderma cell, or a yeast cell (e.g., a methylotrophic yeast cell)). As used herein, “host cell” refers to the particular cell into which the nucleic acid is introduced and also includes the progeny of such a cell that carry the vector. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A construct or vector containing a nucleic acid (e.g., a nucleic acid that encodes a polypeptide) also is provided. Constructs or vectors, including expression constructs or vectors, are commercially available or can be produced by recombinant DNA techniques routine in the art. A construct or vector containing a nucleic acid can have expression elements operably linked to such a nucleic acid, and further can include sequences such as those encoding a selectable marker (e.g., an antibiotic resistance gene). A construct or vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides include those that can be used in purification of the encoded polypeptide (e.g., 6xHis tag, glutathione S-transferase (GST)).

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2X SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, Calif.).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Methods are described herein that can be used to generate a strain that lacks sequences for selection (i.e., that lacks a selectable marker). These methods include using a circular plasmid DNA vector and a linear DNA sequence; the circular plasmid DNA vector contains a selection marker and an origin of DNA replication (also known as an autonomously replicating sequence (ARS)), and the linear DNA sequence contains sequences for integration into the genome (e.g., the Pichia genome) by homologous recombination. The linear DNA molecule additionally can include nucleic acid sequences encoding one or more proteins of interest such as, without limitation, an ALAS, a heme-binding protein, or a third protein (e.g., a transcription factor or a protein involved in heme biosynthesis).

Cells (e.g., Pichia cells) can be transformed with both DNA molecules and the transformants selected by the presence of the selectable marker on the circular plasmid. Transformants then can be screened for integration of the linear DNA molecule into the genome using, for example, PCR. Once transformants with the correct integration of the marker-free linear DNA molecule are identified, the cells can be grown in the absence of selection for the circular plasmid. Because the marker-bearing plasmid is not stably maintained in the absence of selection, the plasmid is lost, often very quickly, after selection is relaxed. The resulting strain carries the integrated linear DNA in the absence of heterologous sequences for selection. Therefore, this approach can be used to construct strains (e.g., Pichia strains) that lack a selectable marker (e.g., a heterologous selection marker) with little to no impact on recombinant protein yield.

In accordance with the present disclosure, there may be employed conventional molecular biology, microbiology, biochemical, and recombinant DNA techniques within the skill of the art. Such techniques are explained fully in the literature. The materials and methods of the disclosure will be further described in the following examples, which do not limit the scope of the methods and compositions of matter described in the claims.

The materials and methods of the disclosure will be further described in the following examples, which do not limit the scope the claims.

### EXEMPLARY EMBODIMENTS

Embodiment 1 is a cell comprising:
a first exogenous nucleic acid construct comprising a nucleotide sequence encoding an aminolevulinate synthase (ALAS) protein operably linked to a first promoter element, wherein the ALAS comprises at least a first heme responsive motif (HRM), and wherein the ALAS comprises a mutation in the first HRM; and
a second exogenous nucleic acid construct comprising a nucleotide sequence encoding a heme-binding protein,
wherein the second exogenous nucleic acid construct comprising a nucleotide sequence encoding the heme-binding protein is operably linked to the first promoter element or is operably linked to a second promoter element.

Embodiment 2 is the cell of embodiment 1, wherein the cell is a fungal cell. Embodiment 3 is the cell of embodiment 2, wherein the cell is an Aspergillus or Trichoderma cell.

Embodiment 4 is the cell of any one of embodiments 1-3, wherein the cell is a yeast cell.

Embodiment 5 is the cell of embodiment 4, wherein the yeast cell is a methylotrophic yeast cell.

Embodiment 6 is the cell of embodiment 5, wherein the methylotrophic yeast cell is a Pichia cell, a Candida cell, a Hansenula cell, or a Torulopsis cell.

Embodiment 7 is the cell of any one of embodiments 5-6, wherein the methylotrophic yeast cell is a Pichia methanolica cell, a Pichia pastoris cell, a Candida boidinii cell, or a Hansenula polymorpha cell.

Embodiment 8 is the cell of any one of embodiments 5-7, wherein the methylotrophic yeast cell is a Pichia pastoris cell.

Embodiment 9 is the cell of any one of embodiments 1-8, wherein the mutation in the first HRM is a mutation from a cysteine to a different amino acid.

Embodiment 10 is the cell of any one of embodiments 1-9, wherein the ALAS protein comprises a second HRM, and wherein the ALAS protein comprises a mutation in the second HRM.

Embodiment 11 is the cell of embodiment 10, wherein the mutation in the second HRM is a mutation from a cysteine to a different amino acid.

Embodiment 12 is the cell of embodiment 10 or embodiment 11, wherein the different amino acid is the same for the mutation in the first HRM and the mutation in the second HRM.

Embodiment 13 is the cell of embodiment 10 or embodiment 11, wherein the different amino acid is not the same for the mutation in the first HRM and the mutation in the second HRM.

Embodiment 14 is the cell of any one of embodiments 10-13, wherein the ALAS protein comprises a third HRM, and wherein the ALAS protein comprises a mutation in the third HRM.

Embodiment 15 is the cell of embodiment 14, wherein the mutation in the third HRM is a mutation from a cysteine to a different amino acid.

Embodiment 16 is the cell of embodiment 15, wherein the different amino acid is the same for the mutation in the first HRM, the mutation in the second HRM, and the mutation in the third HRM.

Embodiment 17 is the cell of any one of embodiments 9-16, wherein the different amino acid is selected from the group consisting of arginine, histidine, lysine, serine, threonine, asparagine, glutamine, selenocysteine, glycine, proline, alanine, isoleucine, leucine, methionine, glutamic acid, aspartic acid, phenylalanine, tryptophan, tyrosine, and valine.

Embodiment 18 is the cell of any one of embodiments 9-16, wherein the different amino acid is selected from a nonpolar aliphatic amino acid, an aromatic amino acid, a polar uncharged amino acid, or a positively charged amino acid.

Embodiment 19 is the cell of embodiment 18, wherein the nonpolar aliphatic amino acid is selected from the group consisting of glycine, proline, alanine, isoleucine, leucine, methionine, and valine.

Embodiment 20 is the cell of embodiment 18, wherein the aromatic amino acid is selected from the group consisting of phenylalanine, tryptophan, and tyrosine.

Embodiment 21 is the cell of embodiment 18, wherein the polar uncharged amino acid is selected from the group consisting of a polar uncharged amino acid serine, threonine, asparagine, or glutamine.

Embodiment 22 is the cell of embodiment 18, wherein the positively charged amino acid is selected from the group consisting of arginine, histidine, and lysine.

Embodiment 23 is the cell of any one of embodiments 9-22, wherein the different amino acid is serine.

Embodiment 24 is the cell of any one of embodiments 9-22, wherein the different amino acid is alanine.

Embodiment 25 is the cell of any one of embodiments 9-22, wherein the different amino acid is phenylalanine.

Embodiment 26 is the cell of any one of embodiments 9-22, wherein the different amino acid is histidine.

Embodiment 27 is the cell of any one of embodiments 1-26, wherein the first HRM is HRM1.

Embodiment 28 is the cell of any one of embodiments 10-27, wherein the second HRM is HRM2.

Embodiment 29 is the cell of any one of embodiments 1-26, wherein the first HRM is HRM2.

Embodiment 30 is the cell of any one of embodiments 10-27, wherein the second HRM is HRM1.

Embodiment 31 is the cell of any one of embodiments 1-30, wherein the first exogenous nucleic acid construct comprises a nucleic acid sequence having at least 90% sequence identity to the nucleic acid sequence in SEQ ID NO: 28.

Embodiment 32 is the cell of any one of embodiments 1-30, wherein the first exogenous nucleic acid construct comprises a nucleic acid sequence having at least 95% sequence identity to the nucleic acid sequence in SEQ ID NO: 28.

Embodiment 33 is the cell of any one of embodiments 1-30, wherein the first exogenous nucleic acid construct comprises the nucleic acid sequence in SEQ ID NO: 30.

Embodiment 34 is the cell of any one of embodiments 1-32, wherein the ALAS protein comprises an amino acid sequence having at least 90% sequence identity to the amino acid sequence in SEQ ID NO: 29.

Embodiment 35 is the cell of any one of embodiments 1-32, wherein the ALAS protein comprises an amino acid sequence having at least 95% sequence identity to the amino acid sequence in SEQ ID NO: 29.

Embodiment 36 is the cell of any one of embodiments 1-35, wherein the ALAS protein comprises the amino acid sequence in SEQ ID NO: 31.

Embodiment 37 is the cell of any one of embodiments 1-36, wherein the heme-binding protein is selected from the group consisting of a globin, a cytochrome, a cytochrome c oxidase, a ligninase, a catalase, and a peroxidase.

Embodiment 38 is the cell of any one of embodiments 1-36, wherein the heme-binding protein is selected from the group consisting of an androglobin, a chlorocruorin, a cytoglobin, an erythrocruorin, a flavohemoglobin, a globin E, a globin X, a globin Y, a hemoglobin, a histoglobin, a leghemoglobin, a myoglobin, a neuroglobin, a non-symbiotic hemoglobin, a protoglobin, and a truncated hemoglobin.

Embodiment 39 is the cell of any one of embodiments 1-36, wherein the heme-binding protein is a non-symbiotic hemoglobin.

Embodiment 40 is the cell of any one of embodiments 1-36, wherein the heme-binding protein is a leghemoglobin.

Embodiment 41 is the cell of any one of embodiments 1-40, wherein the heme-binding protein comprises an amino acid sequence having at least 90% sequence identity to an amino acid sequence in any one of SEQ ID NOs: 1-27.

Embodiment 42 is the cell of any one of embodiments 1-41, further comprising a third nucleic acid construct comprising a nucleotide sequence encoding a transcription factor, wherein the third nucleic acid construct is operably linked to the first promoter element, the second promoter element, or a third promoter element.

Embodiment 43 is the cell of embodiment 42, wherein the first promoter element comprises a recognition sequence for the transcription factor.

Embodiment 44 is the cell of any one of embodiments 42 or 43, wherein the second exogenous nucleic acid construct is operably linked to a second promoter element, and wherein the second promoter element comprises a recognition sequence for the transcription factor.

Embodiment 45 is the cell any one of embodiments 42 or 43, wherein the third nucleic acid construct is operably linked to the third promoter element, and wherein the third promoter element comprises a recognition sequence for the transcription factor.

Embodiment 46 is the cell of any one of embodiments 1-45, further comprising a fourth nucleic acid construct comprising a nucleotide sequence encoding a protein involved in heme biosynthesis, wherein the fourth nucleic acid construct is operably linked to the first promoter element, the second promoter element, the third promoter element, or a fourth promoter element.

Embodiment 47 is the cell of embodiment 46, wherein the protein involved in heme biosynthesis is selected from the group consisting of ALA dehydratase, porphobilinogen deaminase, UPG III synthase, UPG III decarboxylase, CPG oxidase, PPG oxidase, and ferrochelatase.

Embodiment 48 is the cell of any one of embodiments 1-47, wherein the first exogenous nucleic acid construct is a heterologous nucleic acid construct.

Embodiment 49 is the cell of any one of embodiments 1-48, wherein the second exogenous nucleic acid construct is a heterologous nucleic acid construct.

Embodiment 50 is the cell of any one of embodiments 1-47, wherein the heme-binding protein is an exogenous heme-binding protein.

Embodiment 51 is the cell of any one of embodiments 1-47 or 50, wherein the heme-binding protein is a heterologous heme-binding protein.

Embodiment 52 is a method of producing a heme-binding protein in a cell comprising:
expressing a first exogenous nucleic acid construct comprising a nucleotide sequence encoding an aminolevulinate synthase (ALAS) protein operably linked to a first promoter element, wherein the ALAS comprises at least a first heme responsive motif (HRM), and wherein the ALAS comprises a mutation in the first HRM; and
expressing a second exogenous nucleic acid construct comprising a nucleotide sequence encoding a heme-binding protein,
wherein the second exogenous nucleic acid construct comprising a nucleotide sequence encoding the heme-binding protein is operably linked to the first promoter element or is operably linked to a second promoter element.

Embodiment 53 is the method of 52, wherein the method produces the heme-binding protein in a titer that is at least 5% greater than a corresponding method lacking the first exogenous nucleic acid construct.

Embodiment 54 is the method of embodiment 52 or embodiment 53, wherein the method produces the heme-binding protein in a titer that is at least 10% greater than a corresponding method lacking the first exogenous nucleic acid construct.

Embodiment 55 is the method of embodiment 52 or embodiment 53, wherein the method produces the heme-binding protein in a titer that is at least 15% greater than a corresponding method lacking the first exogenous nucleic acid construct.

Embodiment 56 is the method of embodiment 52 or embodiment 53, wherein the method produces the heme-binding protein in a titer that is at least 20% greater than a corresponding method lacking the first exogenous nucleic acid construct.

Embodiment 57 is the method of embodiment 52, wherein the method produces the heme-binding protein in a titer that is at least 5% greater than a corresponding method lacking the mutation in the first HRM.

Embodiment 58 is the method of embodiment 52, wherein the method produces the heme-binding protein in a titer that is at least 10% greater than a corresponding method lacking the mutation in the first HRM.

Embodiment 59 is the method of embodiment 52, wherein the method produces the heme-binding protein in a titer that is at least 15% greater than a corresponding method lacking the mutation in the first HRM.

Embodiment 60 is the method of embodiment 52, wherein the method produces the heme-binding protein in a titer that is at least 20% greater than a corresponding method lacking the mutation in the first HRM.

Embodiment 61 is the method of any one of embodiments 52-60, wherein the method is carried out in the absence of added methanol.

Embodiment 62 is the method of any one of embodiments 52-61, wherein the cell is a fungal cell.

Embodiment 63 is the method of embodiment 62, wherein the cell is an Aspergillus or Trichoderma cell

Embodiment 64 is the method of any one of embodiments 62-63, wherein the cell is a yeast cell.

Embodiment 65 is the method of embodiment 64, wherein the yeast cell is a methylotrophic yeast cell.

Embodiment 66 is the method of embodiment 65, wherein the methylotrophic yeast cell is a Pichia cell, a Candida cell, a Hansenula cell, or a Torulopsis cell.

Embodiment 67 is the method of any one of embodiments 65-66, wherein the methylotrophic yeast cell is a Pichia methanolica cell, a Pichia pastoris cell, a Candida boidinii cell, or a Hansenula polymorpha cell.

Embodiment 68 is the method of any one of embodiments 51-53, wherein the methylotrophic yeast cell is a Pichia pastoris cell.

Embodiment 69 is the method of any one of embodiments 52-68, wherein the mutation in the first HRM is a mutation from a cysteine to a different amino acid.

Embodiment 70 is the method of any one of embodiments 52-69, wherein the ALAS protein comprises a second HRM, and wherein the ALAS protein comprises a mutation in the second HRM.

Embodiment 71 is the method of embodiment 70, wherein the method produces the heme-binding protein in a titer that is at least 5% greater than a corresponding method lacking the mutations in the first HRM and second HRM.

Embodiment 72 is the method of embodiment 70, wherein the method produces the heme-binding protein in a titer that is at least 10% greater than a corresponding method lacking the mutations in the first HRM and second HRM.

Embodiment 73 is the method of embodiment 70, wherein the method produces the heme-binding protein in a titer that is at least 15% greater than a corresponding method lacking the mutations in the first HRM and second HRM.

Embodiment 74 is the method of embodiment 70, wherein the method produces the heme-binding protein in a titer that is at least 20% greater than a corresponding method lacking the mutations in the first HRM and second HRM.

Embodiment 75 is the method of any one of embodiments 70-74, wherein the mutation in the second HRM is a mutation from a cysteine to a different amino acid.

Embodiment 76 is the method of any one of embodiments 70-75, wherein the different amino acid is the same for the mutation in the first HRM and the mutation in the second HRM.

Embodiment 77 is the method of any one of embodiments 70-75, wherein the different amino acid is not the same for the mutation in the first HRM and the mutation in the second HRM.

Embodiment 78 is the cell of any one of embodiments 70-77, wherein the ALAS protein comprises a third HRM, and wherein the ALAS protein comprises a mutation in the third HRM.

Embodiment 79 is the cell of embodiment 78, wherein the mutation in the third HRM is a mutation from a cysteine to a different amino acid.

Embodiment 80 is the cell of embodiment 79, wherein the different amino acid is the same for the mutation in the first HRM, the mutation in the second HRM, and the mutation in the third HRM.

Embodiment 81 is the method any one of embodiments 69-80, wherein the different amino acid is selected from the group consisting of arginine, histidine, lysine, serine, threonine, asparagine, glutamine, selenocysteine, glycine, proline, alanine, isoleucine, leucine, methionine, aspartic acid, glutamic acid, phenylalanine, tryptophan, tyrosine, and valine.

Embodiment 82 is the method of any one of embodiments 69-80, wherein the different amino acid is selected from a nonpolar aliphatic amino acid, an aromatic amino acid, a polar uncharged amino acid, or a positively charged amino acid.

Embodiment 83 is the method of embodiment 82, wherein the nonpolar aliphatic amino acid is selected from the group consisting of glycine, proline, alanine, isoleucine, leucine, methionine, and valine.

Embodiment 84 is the method of embodiment 82, wherein the aromatic amino acid is selected from the group consisting of phenylalanine, tryptophan, and tyrosine.

Embodiment 85 is the cell of embodiment 82, wherein the polar uncharged amino acid is selected from the group consisting of a polar uncharged amino acid serine, threonine, asparagine, or glutamine.

Embodiment 86 is the method of embodiment 82, wherein the positively charged amino acid is selected from the group consisting of arginine, histidine, and lysine.

Embodiment 87 is the method of any one of embodiments 69-86, wherein the different amino acid is serine.

Embodiment 88 is the method of any one of embodiments 69-86, wherein the different amino acid is alanine.

Embodiment 89 is the method of any one of embodiments 69-86, wherein the different amino acid is phenylalanine.

Embodiment 90 is the method of any one of embodiments 69-86, wherein the different amino acid is histidine.

Embodiment 91 is the cell of any one of embodiments 52-90, wherein the first HRM is HRM1.

Embodiment 92 is the cell of any one of embodiments 70-91, wherein the second HRM is HRM2.

Embodiment 93 is the cell of any one of embodiments 52-90, wherein the first HRM is HRM2.

Embodiment 94 is the cell of any one of embodiments 70-91, wherein the second HRM is HRM1.

Embodiment 95 is the method of any one of embodiments 52-94, wherein the first exogenous nucleic acid construct comprises a nucleic acid sequence having at least 90% sequence identity to the nucleic acid sequence in SEQ ID NO: 28.

Embodiment 96 is the method of any one of embodiments 52-95, wherein the first exogenous nucleic acid construct comprises a nucleic acid sequence having at least 95% sequence identity to the nucleic acid sequence in SEQ ID NO: 28.

Embodiment 97 is the method of any one of embodiments 52-96, wherein the first exogenous nucleic acid construct comprises the nucleic acid sequence in SEQ ID NO: 30.

Embodiment 98 is the method of any one of embodiments 52-97, wherein the ALAS protein comprises an amino acid sequence having at least 90% sequence identity to the amino acid sequence in SEQ ID NO: 29.

Embodiment 99 is the method of any one of embodiments 52-98, wherein the ALAS protein comprises an amino acid sequence having at least 95% sequence identity to the amino acid sequence in SEQ ID NO: 29.

Embodiment 100 is the method of any one of embodiments 52-99, wherein the ALAS protein comprises the amino acid sequence in SEQ ID NO: 31.

Embodiment 101 is the method of any one of embodiments 52-100, wherein the heme-binding protein is selected from the group consisting of a globin, a cytochrome, a cytochrome c oxidase, a ligninase, a catalase, and a peroxidase.

Embodiment 102 is the method of any one of embodiments 52-100, wherein the heme-binding protein is selected from the group consisting of an androglobin, a chlorocruorin, a cytoglobin, an erythrocruorin, a flavohemoglobin, a globin E, a globin X, a globin Y, a hemoglobin, a histoglobin, a leghemoglobin, a myoglobin, a neuroglobin, a non-symbiotic hemoglobin, a protoglobin, and a truncated hemoglobin.

Embodiment 103 is the method of any one of embodiments 52-100, wherein the heme-binding protein is a non-symbiotic hemoglobin.

Embodiment 104 is the method of any one of embodiments 52-100, wherein the heme-binding protein is a leghemoglobin.

Embodiment 105 is the method of any one of embodiments 52-100, wherein the heterologous heme-binding protein comprises an amino acid sequence having at least 90% sequence identity to an amino acid sequence in any one of SEQ ID NOs: 1-27.

Embodiment 106 is the method of any one of embodiments 52-105, further comprising expressing a third nucleic acid construct comprising a nucleotide sequence encoding a transcription factor, wherein the third nucleic acid construct is operably linked to the first promoter element, the second promoter element, or a third promoter element.

Embodiment 107 is the method of embodiment 106, wherein the first promoter element comprises a recognition sequence for the transcription factor.

Embodiment 108 is the method of embodiment 107, wherein the second exogenous nucleic acid construct is operably linked to a second promoter element, and wherein the second promoter element comprises a recognition sequence for the transcription factor.

Embodiment 109 is the method of any one of embodiments 107-108, wherein the third nucleic acid construct is operably linked to the third promoter element, and wherein the third promoter element comprises a recognition sequence for the transcription factor.

Embodiment 110 is the method of any one of embodiments 52-109, further comprising expressing a fourth nucleic acid construct comprising a nucleotide sequence encoding a protein involved in heme biosynthesis, wherein the fourth nucleic acid construct is operably linked to the first promoter element, the second promoter element, the third promoter element, or a fourth promoter element.

Embodiment 111 is the method of embodiment 110, wherein the protein involved in heme biosynthesis is selected from the group consisting of ALA dehydratase, porphobilinogen deaminase, UPG III synthase, UPG III decarboxylase, CPG oxidase, PPG oxidase, and ferrochelatase.

Embodiment 112 is the method of any one of embodiments 52-111, wherein the first exogenous nucleic acid construct is a heterologous nucleic acid construct.

Embodiment 113 is the method of any one of embodiments 52-112, wherein the second exogenous nucleic acid construct is a heterologous nucleic acid construct.

Embodiment 114 is the method of any one of embodiments 52-113, wherein the heme-binding protein is an exogenous heme-binding protein.

Embodiment 115 is the method of any one of embodiments 52-111 or 114, wherein the heme-binding protein is a heterologous heme-binding protein.

### EXAMPLES

### Example 1

### Polymerase Chain Reaction

Genes of interest were amplified from genomic DNA or plasmid DNA templates using Phusion High-fidelity PCR master mix (New England Biolabs, Cat #M0531), 0.6 μM each of forward and reverse primers and 10-50 ng of template DNA. The reaction conditions were as follows:

| | | | |
|---|---|---|---|
| 1 cycle | Initial | 98° C. | 1 min |
| | Denaturation | | |
| 25 cycles | Denaturation | 98° C. | 10 sec |
| | Annealing | 60° C. | 20 sec |
| | Extension | 72° C. | 30 sec per kb |
| 1 cycle | Final Extension | 72° C. | 5 min |
| | Hold | 4° C. | Forever |

Pichia pastoris ALAS gene (KEGG identifier PAS_chr2-1_0716, which can be accessed at the Kyoto Encyclopedia of Genes and Genomes (KEGG) website) was amplified using primers (ATGGAGTTTGTCGCCCGTCAG; SEQ ID NO: 65) and (CTACAATCTGACTCCTGATGAGGTTTC; SEQ ID NO: 66) from genomic DNA. In the examples, wtALAS denotes this native sequence of ALAS (SEQ ID NO: 28).

### Example 2

### Cloning of ALAS and Mutagenesis

PCR product was purified using NucleoSpin Gel and PCR Clean-Up (Takara Bio, Cat #740609) and cloned in pCR-BluntII-TOPO vector using Zero Blunt TOPO PCR Cloning Kit (Thermo Fisher Scientific, Cat #K280020) following the manufacturer's recommendation. Site directed mutagenesis was carried out on the resulting vector (40 ng of purified plasmid) using two sets of primers (set1: CGTCAGTCCATGAATGCCTCTCCCTTTGTCAGGTCAACTTC; SEQ ID NO: 37 and GAAGTTGACCTGACAAAGGGAGAGGCATTCATGGACTGACG; SEQ ID NO: 38, set2: GCTGCTACTGCTAGTCATTCTCCCGTGGTTGGCCCTG; SEQ ID NO: 39 and CAGGGCCAACCACGGGAGAATGACTAGCAGTAGCAGC; SEQ ID NO: 40) and QuikChange II XL Site-Directed Mutagenesis Kit (Agilent, Cat #200521) to create mutALAS carrying mutations of cysteine residues at positions 12 and 39 to serine residues (SEQ ID NO: 30). In the Examples, mutALAS specifically denotes these two mutations unless specified otherwise. The reaction conditions were as follows:

| | | | |
|---|---|---|---|
| 1 cycle | Initial | 95° C. | 1 min |
| | Denaturation | | |
| 18 cycles | Denaturation | 95° C. | 50 sec |
| | Annealing | 60° C. | 50 sec |
| | Extension | 68° C. | 11 min |
| 1 cycle | Final Extension | 68° C. | 7 min |
| | Hold | 4° C. | Forever |

Single cysteine to serine mutation, C12S or C39S was performed by site directed mutagenesis on vector carrying wtALAS using primer set1 (SEQ ID NO: 37, SEQ ID NO: 38) and set2 (SEQ ID NO: 39, SEQ ID NO: 40) respectively.

### Example 3

### Construction of ALAS (wild-type or mutated, C12S, C39S) Gene Integration Cassette

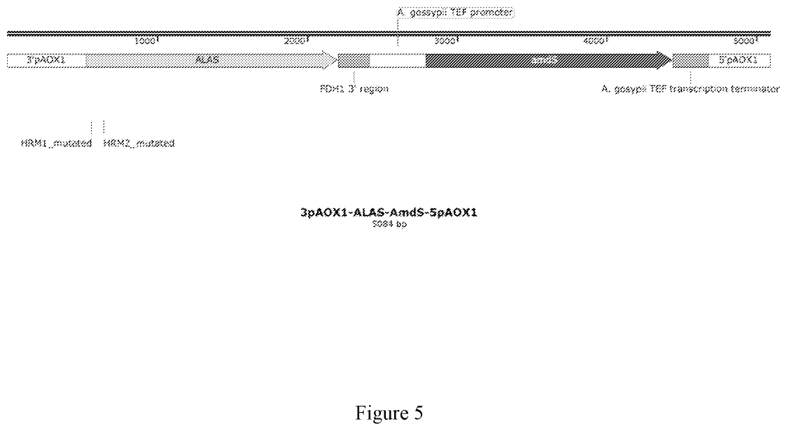

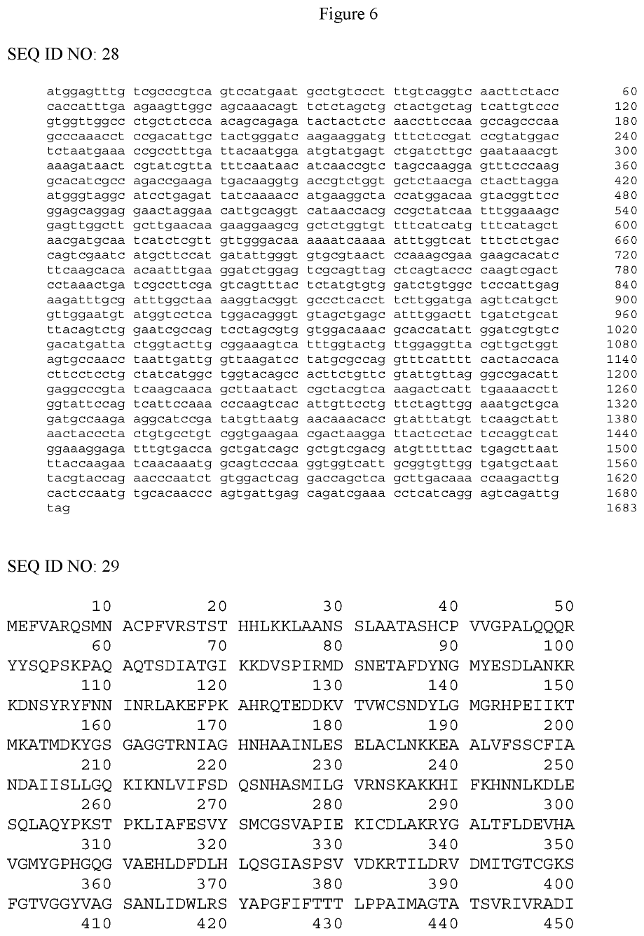

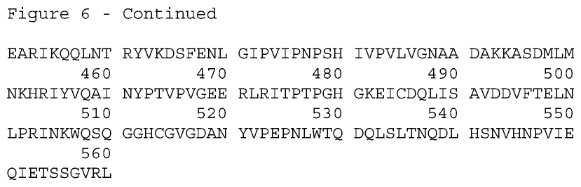

Integration cassette of wtALAS or mutALAS was designed such that gene integration occurred under the methanol-inducible alcohol oxidase 1 (AOX1) promoter element from Pichia pastoris and before the translation stop signal immediately followed by the transcription terminator sequence from the Pichia pastoris FDH1 gene. The linear constructs contained the 3′ half of the promoter element, followed by ALAS gene (wild-type or mutant), followed by the FDH1 transcription terminator. This was immediately followed by the selection cassette containing the pTEF promoter element from Ashbya gossypii, the acetamidase gene (amdS) from Aspergillus nidulans and the TEF terminator from Ashbya gossypii. Finally, the construct contained the 5′ half of the promoter element (See, e.g., FIG. 5).

Overlapping PCR was used to create the linear constructs [3′pAOX1-ALAS (wt/mut)-FDH1tt-pTEF-amdS-TEFtt-5′pAOX1). Primers Used to Amplify the Linear Constructs:

| Overlapping | | SEQ |
|---|---|---|
| PCR | Primer sequences | ID NO: |
| PCR1 | AAACGCTGTCTTGGAACCTAATATGAC | 41 |
| | GACGGGCGACAAACTCCATCGTTTCGA | 42 |
| | ATAATTAGTTG | |
| PCR2 | CAACTAATTATTCGAAACGATGGAGTTT | 43 |
| | GTCGCCCGTCAG | |
| | AATTAAATACATTTCAACTACAATCTGA | 44 |
| | CTCCTGATGAGGTTTCG | |
| PCR3 | CCTCATCAGGAGTCAGATTGTAGTTGAA | 45 |
| | ATGTATTTAATTTG | |
| | AAACTGTCAGTTTTGGGCCATTTG | 46 |

Individual PCR reaction conditions were performed as indicated above. PCR products were purified using NucleoSpin Gel and PCR Clean-Up (Takara Bio, Cat #740609). The final overlapping PCR product was performed using 0.5 U Platinum Pfx DNA Polymerase (Thermo Fisher Scientific, Cat#11708039), 1×amplification buffer, 0.3 mM dNTPs and 1 mM MgCl₂ by mixing the three purified PCR amplicons in an equimolar ratio in a 2-part PCR reaction. The PCR conditions were:

### Part 1:

| | | | |
|---|---|---|---|
| 1 cycle | Initial | 94° C. | 2 min |
| | Denaturation | | |
| 15 cycles | Denaturation | 94° C. | 15 sec |
| | Annealing | 58° C. | 30 sec |
| | Extension | 68° C. | 5 min |
| | Hold | 4° C. | |

Following part 1, the reaction mixes were spiked with primers AAACGCTGTCTTGGAACCTAATATGAC (SEQ ID NO: 41) and AAACTGTCAGTTTTGGGCCATTTG (SEQ ID NO: 46) (0.3 uM final concentration) and 1.5U Pfx and continued to part 2.

### Part 2:

| | | | |
|---|---|---|---|
| 1 cycle | Initial | 94° C. | 2 min |
| | Denaturation | | |
| 20 cycles | Denaturation | 94° C. | 15 sec |
| | Annealing | 58° C. | 30 sec |
| | Extension | 68° C. | 5 min |
| | Hold | 4° C. | Forever |

Sequences of exemplary mutant nucleic acids (e.g., SEQ ID NO: 30) and proteins (e.g., SEQ ID NO: 31) are provided in FIG. 7.

Expression plasmids of wtALAS and mutALAS were constructed in an autonomous replicating vector (panARS) under a modified pAOX1 followed by the transcription terminator sequence from the Pichia pastoris FDH1 gene. The vector conferred resistance to G418 (Geneticin). Cloning of inserts GFP, wtALAS and mutALAS in the vector was performed using Gibson Assembly Master Mix (New England Biolabs, catalog # E2611L) following manufacturer's recommendation.

### Example 4

### Preparation of P. pastoris Transformation-Competent Cells

Selected strains of P. pastoris (K. phaffii) were grown to mid-exponential growth phase (˜2 OD) in 25 ml YPD medium. Cells were collected by centrifugation at 930×g for 15 minutes. The cell pellet was resuspended in 2 ml of a solution of 80% YPD and 200 mM HEPES, pH 6.8. 75 μl of 1 M DTT was added. The resuspended cell pellet was mixed at 100 rpm at 30° C. for 25 minutes. A 40 ml volume of ice cold, sterile water was added to the suspension, and the cells were collected by centrifugation at 1125×g for 15 minutes and placed on ice. The cell pellet was resuspended in 40 ml ice cold water and collected as before for two additional wash steps. The cell pellet was then resuspended in 20 ml of ice cold 1 M sorbitol and collected by centrifugation as before. The final cell pellet was suspended in 0.3 ml ice cold, sterile 1 M sorbitol, aliquoted and frozen at −80° C.

### Example 5

### Transformation into P. pastoris

For genome integration, 100-300 ng of linearized DNA was transformed into 30 μl of electrocompetent P. pastoris cells using a 1 mm gap GenePulser cuvette (Bio-Rad, Cat# 1652083) with a GenePulser (Bio-Rad) set at 1.15 kV. 1 ml of YPD/IM sorbitol (1:1 vol/vol) was added immediately to the cells. The cells were allowed to recover for 3 h at 30° C. with shaking at 100 rpm. 100 μl of the recovery mixture was plated on yeast carbon base plates containing 5 mM acetamide (Teknova, Cat#Y5216). Plates were incubated at 30° C. for 48 hours. Individual clones were streaked onto yeast carbon base plates containing acetamide to obtain single colonies and the isolated colonies were used to do colony PCR or gDNA prep to confirm gene integration into the chromosome and sequence the integration construct.

For plasmid DNA transformation, the same steps were followed as for genome integration except that after electroporation and recovery of cells, 50-100 μl of the recovery mixture was plated on YPD plates containing 300 ug/ml G418 (Geneticin).

### Example 6

### Construction of Strains St2, St3, St5 and St6

A high yielding parent strain (St1) had pre-existing recombinant ALAS under the methanol-inducible strong promoter pAOX1 (alcohol oxidase 1) in addition to other pAOX1-driven heme enzymes, the carbon-responsive transcription factor MxR1 and multiple copies of LegH. A low yielding parent LegH strain (St4) lacked recombinant MxR1, ALAS and other heme enzymes except aminolevulinate dehydratase (ALAD).

Competent St1 cells (Table 1; a high LegH titer strain) were transformed with each of the linear cassettes (for wtALAS and mutALAS) and transformants containing the amdS selection cassette were selected based on their ability to grow on agar plates containing acetamide as the sole nitrogen source. The resulting strains (St2 and St3 with integrated cassette for wtALAS and mutALAS respectively, Table 1) were purified, isolated and the presence of pAOX1 driven wtALAS or mutALAS was verified by colony PCR and sequencing. Similarly, competent St4 cells (a low LegH titer strain) were transformed with linear cassettes for wtALAS and mutALAS to obtain St5 and St6 respectively.

TABLE 1
Strains with LegH integrated in genome

| | 1 extra copy of | 1 extra copy of |
|---|---|---|
| | recombinant | recombinant |
| Parent | wtALAS added | mutALAS added |
| High LegH titer | Strain St2 | Strain St3 |
| strain (Strain St1) | | |
| Low LegH titer | Strain St5 | Strain St6 |
| strain (Strain St4) | | |

### Example 7

### ALAS Gene Copy Number Analysis

ALAS gene copy number in different strains was measured by probe-based qPCR. Briefly, genomic DNA (20 ng) was amplified with 1×PrimeTime gene expression mastermix (Integrated DNA Technologies, Cat# 1055770) and PrimeTime qPCR Probe Assays in a real-time qPCR CFX96 machine (Bio-Rad). qPCR normalization was performed with respect to actin. A double delta Ct analysis method was followed to calculate the relative copy number of gene of interest between strains. Primer and probe sequences were:

| | | SEQ | | SEQ |
|---|---|---|---|---|
| | Primer | ID. | Probe | ID. |
| Gene | sequence | NO. | sequence | NO. |
| ALAS | GCTCTCCAACA | 47 | 6-FAM/AAGCCCAAA/ | 51 |
| | GCAGAGATAC | | Zen/CCTCCGACATTG | |
| | GTCCATACGGA | 48 | CTA/3IABkFQ | |
| | TCGGAGAAAC | | | |
| Actin | AGCAACATCC | 49 | HEX/TCGCCGTAA/ | 52 |
| | CTGATTCCG | | Zen/GTTCTTGGTT | |
| | ATGCGTACCTT | 50 | TAGACGTTC/ | |
| | CAATCCTGG | | 3IABkFQ | |

The engineered strains (Table 1) contained identical number of extra ALAS gene copies, either one copy of wtALAS or mutALAS, as measured by qPCR of ALAS normalized to actin levels. Hence, ALAS gene dosage was ruled out for any difference in the phenotype of the resulting strains (e.g., St2 vs St3 and St5 vs St6).

### Example 8

### PCR Detection of Heme Enzyme, MxR1, LegH and Mb Genes

Strain characterization was done by PCR for pAOX1-driven recombinant heme biogenesis pathway enzyme, MxR1 and LegH genes. Forward primer sequence for PCR was TAGCGCAGTCTCTCTATCGCTTC (SEQ ID NO: 53) specific to pAOX1. Reverse primer was specific to each gene of interest as shown below.

| | | | SEQ |
|---|---|---|---|
| | | Amplicon | ID |
| Gene | Reverse Primer | size | NO: |
| 5-aminolevulinate | CACTGGGTTG | 1995 bp | 54 |
| synthase | TGCACATTGG | | |
| Delta- | ACAATATTCT | 1268 bp | 55 |
| aminolevulinate | TCTCTGCCGC | | |
| dehydratase | | | |
| Phorphobilinogen | TTGATCTCGTC | 1358 bp | 56 |
| deaminase | AAGAATGCG | | |
| Uroporphyrinogen | TAGGTGCCACAA | 1102 bp | 57 |
| III synthase | CTTTTGGTTTC | | |
| Uroporphyrinogen | GATCCAATGCGA | 1430 bp | 58 |
| decarboxylase | TGACATTCTTGT | | |
| Coproporphyrinogen | ACCTGCAATAAC | 1301 bp | 59 |
| III oxidase | TCCTCTTCTCTG | | |
| Protoporphyrinogen | CCACTGAGGGTA | 2027 bp | 60 |
| oxidase | GCCGAATC | | |
| Ferrochelatase | GGGCTCTGAAAA | 1486 bp | 61 |
| | ACTCTTTTGG | | |
| MxR1 | GCATGTCTCAATAA | 629 bp | 62 |
| | CAGATCTCGACGG | | |
| LegH | AAGCCTCTTGTTTT | 382 bp | 63 |
| | TCTGTAAATGCAC | | |
| Bovine Mb | TGATGGCGTCCGA | 688 bp | 64 |
| | GATGAACTC | | |

Results of these reactions are shown below.

| Recombinant Gene (under pAOX1) | St1 | St2 | St3 | St4 | St5 | St6 | St7 |
|---|---|---|---|---|---|---|---|
| 5-aminolevulinate synthase | x | x | x | — | x | x | x |
| Delta-aminolevulinate dehydratase | x | x | x | x | x | x | x |
| Phorphobilinogen deaminase | x | x | x | — | — | — | x |
| Uroporphyrinogen III synthase | x | x | x | — | — | — | x |
| Uroporphyrinogen decarboxylase | x | x | x | — | — | — | x |
| Coproporphyrinogen III oxidase | x | x | x | — | — | — | x |
| Protoporphyrinogen oxidase | x | x | x | — | — | — | x |
| Ferrochelatase | x | x | x | — | — | — | x |
| MxR1 | x | x | x | — | — | — | x |
| LegH | x | x | x | x | x | x | — |
| Mb (Bovine Myoglobin) | — | — | — | — | — | — | x |

### Example 9

### Shake Flask Cultivation of Strains St4, St5 and St6

The strains were inoculated into growth media (1% yeast extract, 2% peptone, supplemented with 1% glycerol) overnight at 30° C. with shaking at 200 rpm. The next day the overnight cultures were diluted to an OD600 of 0.5-0.7 with YP media supplemented with 1% methanol and 1% dextrose. The cultures were grown for 48 hours and harvested by centrifugation at ˜4000 g for 15 mins at 4° C.

The low LegH titer strain, St4, didn't have a pre-existing copy of recombinant ALAS, unlike St1. Integrating a copy of mutALAS (St6) led to a ˜30% improvement in LegH titer compared to St5 (wtALAS). The titer calculation is based on LegH content as measured by a liquid chromatography method as described in Example 13.

The relative LegH titer is shown in Table 2.

TABLE 2

| | St5 | St6 |
|---|---|---|
| Relative LegH titer | 1.00 | 1.32 |

### Example 10

### 2L Cultivation of Strains St1, St2 and St3

Strains St1, St2 and St3 were grown in 2L fermentation tanks in media containing dextrose as the principal carbon source at 30° C. No methanol was used. In the background of a high LegH titer strain (St1) that contained pre-existing recombinant ALAS, St3 overexpressing mutALAS improved LegH titer by >30% over the parent St1. When compared to wtALAS (in St2), mutALAS (in St3) resulted in a 20% improvement in LegH titer. The titer calculation is based on LegH content as measured by a liquid chromatography method as described in Example 13.

The relative LegH titer is shown in Table 3.

TABLE 3

| | St1 | St2 | St3 |
|---|---|---|---|
| Relative LegH titer | 1.00 | 1.10 | 1.37 |

Hence, mutating both the ALAS HRMs and overexpressing the mutated ALAS (mutALAS) in Pichia strains with different LegH titer led to further improvement in LegH titer. Furthermore, this suggested that mutALAS improved LegH titer independent of the strain genetic makeup (MxR1 and heme enzymes other than ALAS), and presence of methanol.

### Example 11

### MutALAS Improved Heme Levels

A quantitative assay for total heme based on reversed-phase high-performance liquid chromatography indicated that strains with mutALAS accumulated more heme levels than wtALAS containing strains. Shown in Table 4 is heme quantification in strain St3 (mutALAS) vs strain St2 (wtALAS). Additionally, mutALAS increased heme loading in multiple strains.

TABLE 4

| | St2 | St3 |
|---|---|---|
| Relative heme titer | 1.00 | 2.7 |

### Example 12

### ALAS Protein Levels with MutALAS

It is generally believed that heme regulates ALAS levels in a feedback fashion to regulate its own levels. At the protein level, the ALAS level was higher 3-fold in St3 (mutALAS) compared to St2 (wtALAS) (Table 5) as quantitated by shotgun mass spectrometry, when the strains were grown in 2L fermenter tanks with dextrose.

TABLE 5

| | St2 | St3 |
|---|---|---|
| Relative ALAS titer | 1.00 | 3.3 |

### Example 13

### Quantification of Leghemoglobin

Cell broth samples were pelleted down (at 4000×g, 4° C., 30 min) and decanted. The pellet samples were then diluted four times with lysis buffer (150 mM NaCl, 50 mM Potassium Phosphate, pH 7.4). 300 uL of each resuspension was dispensed into a 96 well deep plate with 120 uL of beads (Zirconium/silica beads (0.5 mm)) per well for cell lysis. The lysis was done with a mini bead beater for 3 minutes, then the plate was cooled down on ice for 5 minutes, and followed with another 2 minutes of bead beating. The plate was then spun down (at 4000×g, 4° C., 30 min). The supernatant was filtered through a 0.2 um filter plate (at 4000×g, 4° C., 60 min).

The filtered lysate was loaded onto a UHPLC with a size-exclusion column (Acquity BEH SEC column, 200 Å, 1.7 um, 4.6×150 mm). Method parameters: 1) Mobile phase: 5 mM NaCl, 50 mM Potassium Phosphate, (pH 7.4); 2) Flow rate: 0.3 mL/min; 3) Injection volume: 10 uL; 4) Run time: 15 min; 5) Sample tray temperature: 4C. A calibration curve was built with a purified LegH standard using absorbance at 280 nm and 415 nm. The quantification was done using peak area with valley-to-valley peak integration method. The absorbance at 280 nm is proportional to the amount of the polypeptide present and the absorbance at 415 nm is proportional to the amount of heme present. Where a peak is seen at the same elution time at both wavelengths, a heme containing protein is detected.

### Example 14

### mutALAS Improved Levels of Bovine Myoglobin

In addition to LegH, the production of bovine myoglobin was evaluated. Strain, St7, was generated by integrating myoglobin cDNA of Bos taurus (NM_173881.2) in a strain containing recombinant copies of heme enzymes and MxR1 (St7 characterization is described in Example 8) integrated under pAOX1.

Three expression plasmids for expression of GFP (control), wtALAS and mutALAS were constructed as described in Example 3. Three strains, St8, St9 and St10 were generated using a method as described in Example 5 by transforming these three plasmids overexpressing GFP, wtALAS and mutALAS respectively in strain S7, as shown in Table 6. Episomal expression of wtALAS or mutALAS was obtained by growing transformants using a method described in Example 9, except that the growth media was supplemented with 300 ug/ml G418.

TABLE 6

| | | | Host strain | Resulting |
|---|---|---|---|---|
| Plasmid | expressing | Host strain | expressing | Strain |
| 1 | GFP | St7 | Mb | St8 |
| 2 | wtALAS | | | St9 |
| 3 | mutALAS | | | St10 |
| | (C12S, | | | |
| | C39S) | | | |

The cells were pelleted, and shotgun proteomics by LC-MS was performed to quantitate Mb. When normalized to the myoglobin level in Strain St8 (GFP), the average myoglobin levels in St9 and St10 were as shown in Table 7.

TABLE 7

| | Normalized |
|---|---|
| Strains | Myoglobin levels |
| St8 (GFP) | 1 |
| St9 (wt ALAS) | 1.60 |
| St10 (mutALAS) | 2.58 |

### Example 15

### Mutations to Other Amino Acids

In addition to cysteine to serine mutations, additional mutations were evaluated. Expression plasmids of mutALAS variants were constructed by gene synthesis and cloning in an autonomous replicating vector (panARS) under a modified pAOX1 followed by the FDH1 transcription terminator sequence. Furthermore, these plasmids were transformed in strain St1, using a method as described in Example 5 and the resulting transformants produced LegH when cultured, using a method described in Example 9, except that the growth media was supplemented with 300 ug/ml G418. The titer of LegH produced by these strains was normalized to the titer of LegH produced by wtALAS, as determined by the method described in Example 13. The results are shown in Table 8.

TABLE 8

| | Normalized |
|---|---|
| Variant | LegH titer fold |
| C12, C39 (wtALAS) | 1 |
| C12S, C39S (mutALAS) | 1.14 |
| C12A, C39A | 1.22 |
| C12D, C39D | 0.91 |
| C12F, C39F | 1.17 |
| C12H, C39H | 1.12 |

### Example 16

### Single Cysteine to Serine Mutants in Pichia pastoris

In addition to evaluating the double mutant (C12S, C39S) of ALAS in P. pastoris, single cysteine to serine mutants were also evaluated. Single mutations were created in wtALAS by site directed mutagenesis as described in Example 2. Expression_plasmids for expression of wtALAS, mutALAS and single ALAS mutants (C12S and C39S) were constructed as described in Example 3 and were transformed in strain St1, using a method as described in Example 5. The resulting transformants produced LegH when cultured, using a method described in Example 9, except that the growth media was supplemented with 300 ug/ml G418. The titer of LegH produced by these strains was normalized to the titer of LegH produced by wtALAS, as determined by the method described in Example 13. The results are shown in Table 9.

TABLE 9

| | Normalized |
|---|---|
| Variant | LegH titer fold |
| C12, C39 (wtALAS) | 1 |
| C12S, C39S | 1.27 |
| C12S | 1.03 |
| C39S | 1.01 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A cell comprising:
a first exogenous nucleic acid construct comprising a nucleotide sequence encoding an aminolevulinate synthase (ALAS) protein operably linked to a first promoter element, wherein the ALAS comprises at least a first heme responsive motif (HRM), and wherein the ALAS comprises a mutation in the first HRM; and
a second exogenous nucleic acid construct comprising a nucleotide sequence encoding a heme-binding protein,
wherein the second exogenous nucleic acid construct comprising a nucleotide sequence encoding the heme-binding protein is operably linked to the first promoter element or is operably linked to a second promoter element.

2. The cell of claim 1, wherein the cell is a fungal cell, or is a yeast cell.

3. The cell of claim 1, wherein the mutation in the first HRM is a mutation from a cysteine to a different amino acid.

4. The cell claim 3, wherein the ALAS protein comprises a second HRM, and wherein the ALAS protein comprises a mutation in the second HRM.

5. The cell of claim 4, wherein the mutation in the second HRM is a mutation from a cysteine to a different amino acid.

6. The cell of claim 5, wherein the different amino acid is the same for the mutation in the first HRM and the mutation in the second HRM.

7. The cell of claim 3, wherein the different amino acid is serine, alanine, phenylalanine, or histidine.

8. The cell of claim 1, wherein the ALAS protein comprises an amino acid sequence having at least 90% sequence identity to the amino acid sequence in SEQ ID NO: 29.

9. The cell of claim 1, wherein the heme-binding protein is selected from the group consisting of a globin, a cytochrome, a cytochrome c oxidase, a ligninase, a catalase, and a peroxidase.

10. A method of producing a heme-binding protein in a cell comprising:
expressing a first exogenous nucleic acid construct comprising a nucleotide sequence encoding an aminolevulinate synthase (ALAS) protein operably linked to a first promoter element, wherein the ALAS comprises at least a first heme responsive motif (HRM), and wherein the ALAS comprises a mutation in the first HRM; and
expressing a second exogenous nucleic acid construct comprising a nucleotide sequence encoding a heme-binding protein,
wherein the second exogenous nucleic acid construct comprising a nucleotide sequence encoding the heme-binding protein is operably linked to the first promoter element or is operably linked to a second promoter element.

11. The method of claim 10, wherein the cell is a fungal cell, or is a yeast cell.

12. The method of claim 10, wherein the mutation in the first HRM is a mutation from a cysteine to a different amino acid.

13. The method claim 12, wherein the ALAS protein comprises a second HRM, and wherein the ALAS protein comprises a mutation in the second HRM.

14. The method of claim 13, wherein the method produces the heme-binding protein in a titer that is at least 5% greater than a corresponding method lacking the mutations in the first HRM and second HRM.

15. The method of claim 13, wherein the method produces the heme-binding protein in a titer that is at least 10% greater than a corresponding method lacking the mutations in the first HRM and second HRM.

16. The method of claim 13, wherein the mutation in the second HRM is a mutation from a cysteine to a different amino acid.

17. The method of claim 16, wherein the different amino acid is the same for the mutation in the first HRM and the mutation in the second HRM.

18. The method of claim 12, wherein the different amino acid is serine, alanine, phenylalanine, or histidine.

19. The method of claim 10, wherein the ALAS protein comprises an amino acid sequence having at least 90% sequence identity to the amino acid sequence in SEQ ID NO: 29.

20. The method of claim 10, wherein the heme-binding protein is selected from the group consisting of a globin, a cytochrome, a cytochrome c oxidase, a ligninase, a catalase, and a peroxidase.


SEQUENCE LISTING (66 sequences)

SEQ ID NO: 1 - protein, 161 aa, Vigna radiata
MTTTLERGFTEEQEALVVKSWNVMKKNSGELGLKFFLKIFEIAPSAQKLFSFLRDSTVPLEQNPKLKPHAVSVFVMTCDSAVQLRKAGKVTVRESNLKKLGATHFRTGVANEHFEVTKFALLETIKEAVPEMWSPAMKNAWGEAYDQLVDAIKYEMKPPSS

SEQ ID NO: 2 - protein, 133 aa, Methylacidiphilum infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLVGDCLLKSIQEYLGQGFTEEAKAAWTKVYGIAAQVMTAE

SEQ ID NO: 3 - protein, 139 aa, Aquifex aeolicus
MLSEETIRVIKSTVPLLKEHGTEITARMYELLFSKYPKTKELFAGASEEQPKKLANAIIAYATYIDRLEELDNAISTIARSHVRRNVKPEHYPLVKECLLQAIEEVLNPGEEVLKAWEEAYDFLAKTLITLEKKLYSQP

SEQ ID NO: 4 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 5 - protein, 162 aa, Hordeum vulgare
MSAAEGAVVFSEEKEALVLKSWAIMKKDSANLGLRFFLKIFEIAPSARQMFPFLRDSDVPLETNPKLKTHAVSVFVMTCEAAAQLRKAGKITVRETTLKRLGGTHLKYGVADGHFEVTRFALLETIKEALPADMWGPEMRNAWGEAYDQLVAAIKQEMKPAE

SEQ ID NO: 6 - protein, 1153 aa, Magnaporthe oryzae
MDGAVRLDWTGLDLTGHEIHDGVPIASRVQVMVSFPLFKDQHIIMSSKESPSRKSSTIGQSTRNGSCQADTQKGQLPPVGEKPKPVKENPMKKLKEMSQRPLPTQHGDGTYPTEKKLTGIGEDLKHIRGYDVKTLLAMVKSKLKGEKLKDDKTMLMERVMQLVARLPTESKKRAELTDSLINELWESLDHPPLNYLGPEHSYRTPDGSYNHPFNPQLGAAGSRYARSVIPTVTPPGALPDPGLIFDSIMGRTPNSYRKHPNNVSSILWYWATIIIHDIFWTDPRDINTNKSSSYLDLAPLYGNSQEMQDSIRTFKDGRMKPDCYADKRLAGMPPGVSVLLIMFNRFHNHVAENLALINEGGRFNKPSDLLEGEAREAAWKKYDNDLFQVARLVTSGLYINITLVDYVRNIVNLNRVDTTWTLDPRQDAGAHVGTADGAERGTGNAVSAEFNLCYRWHSCISEKDSKFVEAQFQNIFGKPASEVRPDEMWKGFAKMEQNTPADPGQRTFGGFKRGPDGKFDDDDLVRCISEAVEDVAGAFGARNVPQAMKVVETMGIIQGRKWNVAGLNEFRKHFHLKPYSTFEDINSDPGVAEALRRLYDHPDNVELYPGLVAEEDKQPMVPGVGIAPTYTISRVVLSDAVCLVRGDRFYTTDFTPRNLTNWGYKEVDYDLSVNHGCVFYKLFIRAFPNHFKQNSVYAHYPMVVPSENKRILEALGRADLFDFEAPKYIPPRVNITSYGGAEYILETQEKYKVTWHEGLGFLMGEGGLKFMLSGDDPLHAQQRKCMAAQLYKDGWTEAVKAFYAGMMEELLVSKSYFLGNNKHRHVDIIRDVGNMVHVHFASQVFGLPLKTAKNPTGVFTEQEMYGILAAIFTTIFFDLDPSKSFPLRTKTREVCQKLAKLVEANVKLINKIPWSRGMFVGKPAKDEPLSIYGKTMIKGLKAHGLSDYDIAWSHVVPTSGAMVPNQAQVFAQAVDYYLSPAGMHYIPEIHMVALQPSTPETDALLLGYAMEGIRLAGTFGSYREAAVDDVVKEDNGRQVPVKAGDRVFVSFVDAARDPKHFPDPEVVNPRRPAKKYIHYGVGPHACLGRDASQIAITEMFRCLFRRRNVRRVPGPQGELKKVPRPGGFYVYMREDWGGLFPFPVTMRVMWDDE

SEQ ID NO: 7 - protein, 530 aa, Fusarium oxysporum
MKGSATLAFALVQFSAASQLVWPSKWDEVEDLLYMQGGFNKRGFADALRTCEFGSNVPGTQNTAEWLRTAFHDAITHDAKAGTGGLDASIYWESSRPENPGKAFNNTFGFFSGFHNPRATASDLTALGTVLAVGACNGPRIPFRAGRIDAYKAGPAGVPEPSTNLKDTFAAFTKAGFTKEEMTAMVACGHAIGGVHSVDFPEIVGIKADPNNDTNVPFQKDVSSFHNGIVTEYLAGTSKNPLVASKNATFHSDKRIFDNDKATMKKLSTKAGFNSMCADILTRMIDTVPKSVQLTPVLEAYDVRPYITELSLNNKNKIHFTGSVRVRITNNIRDNNDLAINLIYVGRDGKKVTVPTQQVTFQGGTSFGAGEVFANFEFDTTMDAKNGITKFFIQEVKPSTKATVTHDNQKTGGYKVDDTVLYQLQQSCAVLEKLPNAPLVVTAMVRDARAKDALTLRVAHKKPVKGSIVPRFQTAITNFKATGKKSSGYTGFQAKTMFEEQSTYFDIVLGGSPASGVQFLTSQAMPSQCS

SEQ ID NO: 8 - protein, 358 aa, Fusarium graminearum
MASATRQFARAATRATRNGFAIAPRQVIRQQGRRYYSSEPAQKSSSAWIWLTGAAVAGGAGYYFYGNSASSATAKVFNPSKEDYQKVYNEIAARLEEKDDYDDGSYGPVLVRLAWHASGTYDKETGTGGSNGATMRFAPESDHGANAGLAAARDFLQPVKEKFPWITYSDLWILAGVCAIQEMLGPAIPYRPGRSDRDVSGCTPDGRLPDASKRQDHLRGIFGRMGFNDQEIVALSGAHALGRCHTDRSGYSGPWTFSPTVLTNDYFRLLVEEKWQWKKWNGPAQYEDKSTKSLMMLPSDIALIEDKKFKPWVEKYAKDNDAFFKDFSNVVLRLFELGVPFAQGTENQRWTFKPTHQE

SEQ ID NO: 9 - protein, 122 aa, Chlamydomonas eugametos
MSLFAKLGGREAVEAAVDKFYNKIVADPTVSTYFSNTDMKVQRSKQFAFLAYALGGASEWKGKDMRTAHKDLVPHLSDVHFQAVARHLSDTLTELGVPPEDITDAMAVVASTRTEVLNMPQQ

SEQ ID NO: 10 - protein, 121 aa, Tetrahymena pyriformis
MNKPQTIYEKLGGENAMKAAVPLFYKKVLADERVKHFFKNTDMDHQTKQQTDFLTMLLGGPNHYKGKNMTEAHKGMNLQNLHFDAIIENLAATLKELGVTDAVINEAAKVIEHTRKDMLGK

SEQ ID NO: 11 - protein, 117 aa, Paramecium caudatum
MSLFEQLGGQAAVQAVTAQFYANIQADATVATFFNGIDMPNQTNKTAAFLCAALGGPNAWTGRNLKEVHANMGVSNAQFTTVIGHLRSALTGAGVAAALVEQTVAVAETVRGDVVTV

SEQ ID NO: 12 - protein, 147 aa, Aspergillus niger
MPLTPEQIKIIKATVPVLQEYGTKITTAFYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQG

SEQ ID NO: 13 - protein, 165 aa, Zea mays
MALAEADDGAVVFGEEQEALVLKSWAVMKKDAANLGLRFFLKVFEIAPSAEQMFSFLRDSDVPLEKNPKLKTHAMSVFVMTCEAAAQLRKAGKVTVRETTLKRLGATHLRYGVADGHFEVTGFALLETIKEALPADMWSLEMKKAWAEAYSQLVAAIKREMKPDA

SEQ ID NO: 14 - protein, 169 aa, Oryza sativa
MALVEGNNGVSGGAVSFSEEQEALVLKSWAIMKKDSANIGLRFFLKIFEVAPSASQMFSFLRNSDVPLEKNPKLKTHAMSVFVMTCEAAAQLRKAGKVTVRDTTLKRLGATHFKYGVGDAHFEVTRFALLETIKEAVPVDMWSPAMKSAWSEAYNQLVAAIKQEMKPAE

SEQ ID NO: 15 - protein, 160 aa, Arabidopsis thaliana
MESEGKIVFTEEQEALVVKSWSVMKKNSAELGLKLFIKIFEIAPTTKKMFSFLRDSPIPAEQNPKLKPHAMSVFVMCCESAVQLRKTGKVTVRETTLKRLGASHSKYGVVDEHFEVAKYALLETIKEAVPEMWSPEMKVAWGQAYDHLVAAIKAEMNLSN

SEQ ID NO: 16 - protein, 147 aa, Pisum sativum
MGFTDKQEALVNSSWESFKQNLSGNSILFYTIILEKAPAAKGLFSFLKDTAGVEDSPKLQAHAEQVFGLVRDSAAQLRTKGEVVLGNATLGAIHVQRGVTDPHFVVVKEALLQTIKKASGNNWSEELNTAWEVAYDGLATAIKKAMT

SEQ ID NO: 17 - protein, 145 aa, Vigna unguiculata
MVAFSDKQEALVNGAYEAFKANIPKYSVVFYTTILEKAPAAKNLFSFLANGVDATNPKLTGHAEKLFGLVRDSAAQLRASGGVVADAALGAVHSQKAVNDAQFVVVKEALVKTLKEAVGDKWSDELGTAVELAYDELAAAIKKAY

SEQ ID NO: 18 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 19 - protein, 154 aa, Sus scrofa
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 20 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 21 - protein, 152 aa, Nicotiana benthamiana
MSSFTEEQEALVVKSWDSMKKNAGEWGLKLFLKIFEIAPSAKKLFSFLKDSNVPLEQNAKLKPHSKSVFVMTCEAAVQLRKAGKVVVRDSTLKKLGATHFKYGVADEHFEVTKFALLETIKEAVPEMWSVDMKNAWGEAFDQLVNAIKTEMK

SEQ ID NO: 22 - protein, 132 aa, Bacillus subtilis
MGQSFNAPYEAIGEELLSQLVDTFYERVASHPLLKPIFPSDLTETARKQKQFLTQYLGGPPLYTEEHGHPMLRARHLPFPITNERADAWLSCMKDAMDHVGLEGEIREFLFGRLELTARHMVNQTEAEDRSS

SEQ ID NO: 23 - protein, 131 aa, Corynebacterium glutamicum
MTTSENFYDSVGGEETFSLIVHRFYEQVPNDDILGPMYPPDDFEGAEQRLKMFLSQYWGGPKDYQEQRGHPRLRMRHVNYPIGVTAAERWLQLMSNALDGVDLTAEQREAIWEHMVRAADMLINSNPDPHA

SEQ ID NO: 24 - protein, 124 aa, Synechocystis sp.
MSTLYEKLGGTTAVDLAVDKFYERVLQDDRIKHFFADVDMAKQRAHQKAFLTYAFGGTDKYDGRYMREAHKELVENHGLNGEHFDAVAEDLLATLKEMGVPEDLIAEVAAVAGAPAHKRDVLNQ

SEQ ID NO: 25 - protein, 183 aa, Synechococcus sp.
MDVALLEKSFEQISPRAIEFSASFYQNLFHHHPELKPLFAETSQTIQEKKLIFSLAAIIENLRNPDILQPALKSLGARHAEVGTIKSHYPLVGQALIETFAEYLAADWTEQLATAWVEAYDVIASTMIEGADNPAAYLEPELTFYEWLDLYGEESPKVRNAIATLTHFHYGEDPQDVQRDSRG

SEQ ID NO: 26 - protein, 118 aa, Nostoc commune
MSTLYDNIGGQPAIEQVVDELHKRIATDSLLAPVFAGTDMVKQRNHLVAFLAQIFEGPKQYGGRPMDKTHAGLNLQQPHFDAIAKHLGERMAVRGVSAENTKAALDRVTNMKGAILNK

SEQ ID NO: 27 - protein, 136 aa, Bacillus megaterium
MREKIHSPYELLGGEHTISKLVDAFYTRVGQHPELAPIFPDNLTETARKQKQFLTQYLGGPSLYTEEHGHPMLRARHLPFEITPSRAKAWLTCMHEAMDEINLEGPERDELYHRLILTAQHMINSPEQTDEKGFSH

SEQ ID NO: 28 - DNA, 1683 bp, Pichia pastoris
  source 1..1683: mol_type = genomic DNA
atggagtttgtcgcccgtcagtccatgaatgcctgtccctttgtcaggtcaacttctacccaccatttgaagaagttggcagcaaacagttctctagctgctactgctagtcattgtcccgtggttggccctgctctccaacagcagagatactactctcaaccttccaagccagcccaagcccaaacctccgacattgctactgggatcaagaaggatgtttctccgatccgtatggactctaatgaaaccgcctttgattacaatggaatgtatgagtctgatcttgcgaataaacgtaaagataactcgtatcgttatttcaataacatcaaccgtctagccaaggagtttcccaaggcacatcgccagaccgaagatgacaaggtgaccgtctggtgctctaacgactacttaggaatgggtaggcatcctgagattatcaaaaccatgaaggctaccatggacaagtacggttccggagcaggaggaactaggaacattgcaggtcataaccacgccgctatcaatttggaaagcgagttggcttgcttgaacaagaaggaagcggctctggtgttttcatcatgtttcatagctaacgatgcaatcatctcgttgttgggacaaaaaatcaaaaatttggtcattttctctgaccagtcgaatcatgcttccatgatattgggtgtgcgtaactccaaagcgaagaagcacatcttcaagcacaacaatttgaaggatctggagtcgcagttagctcagtaccccaagtcgactcctaaactgatcgccttcgagtcagtttactctatgtgtggatctgtggctcccattgagaagatttgcgatttggctaaaaggtacggtgccctcaccttcttggatgaagttcatgctgttggaatgtatggtcctcatggacagggtgtagctgagcatttggactttgatctgcatttacagtctggaatcgccagtcctagcgtggtggacaaacgcaccatattggatcgtgtcgacatgattactggtacttgcggaaagtcatttggtactgttggaggttacgttgctggtagtgccaacctaattgattggttaagatcctatgcgccaggtttcattttcactaccacacttcctcctgctatcatggctggtacagccacttctgttcgtattgttagggccgacattgaggcccgtatcaagcaacagcttaatactcgctacgtcaaagactcatttgaaaaccttggtattccagtcattccaaacccaagtcacattgttcctgttctagttggaaatgctgcagatgccaagaaggcatccgatatgttaatgaacaaacaccgtatttatgttcaagctattaactaccctactgtgcctgtcggtgaagaacgactaaggattactcctactccaggtcatggaaaggagatttgtgaccagctgatcagcgctgtcgacgatgtttttactgagcttaatttaccaagaatcaacaaatggcagtcccaaggtggtcattgcggtgttggtgatgctaattacgtaccagaacccaatctgtggactcaggaccagctcagcttgacaaaccaagacttgcactccaatgtgcacaacccagtgattgagcagatcgaaacctcatcaggagtcagattgtag

SEQ ID NO: 29 - protein, 560 aa, Pichia pastoris
MEFVARQSMNACPFVRSTSTHHLKKLAANSSLAATASHCPVVGPALQQQRYYSQPSKPAQAQTSDIATGIKKDVSPIRMDSNETAFDYNGMYESDLANKRKDNSYRYFNNINRLAKEFPKAHRQTEDDKVTVWCSNDYLGMGRHPEIIKTMKATMDKYGSGAGGTRNIAGHNHAAINLESELACLNKKEAALVFSSCFIANDAIISLLGQKIKNLVIFSDQSNHASMILGVRNSKAKKHIFKHNNLKDLESQLAQYPKSTPKLIAFESVYSMCGSVAPIEKICDLAKRYGALTFLDEVHAVGMYGPHGQGVAEHLDFDLHLQSGIASPSVVDKRTILDRVDMITGTCGKSFGTVGGYVAGSANLIDWLRSYAPGFIFTTTLPPAIMAGTATSVRIVRADIEARIKQQLNTRYVKDSFENLGIPVIPNPSHIVPVLVGNAADAKKASDMLMNKHRIYVQAINYPTVPVGEERLRITPTPGHGKEICDQLISAVDDVFTELNLPRINKWQSQGGHCGVGDANYVPEPNLWTQDQLSLTNQDLHSNVHNPVIEQIETSSGVRL

SEQ ID NO: 30 - DNA, 1683 bp, synthetic construct
  source 1..1683: mol_type = other DNA
atggagtttgtcgcccgtcagtccatgaatgcctctccctttgtcaggtcaacttctacccaccatttgaagaagttggcagcaaacagttctctagctgctactgctagtcattctcccgtggttggccctgctctccaacagcagagatactactctcaaccttccaagccagcccaagcccaaacctccgacattgctactgggatcaagaaggatgtttctccgatccgtatggactctaatgaaaccgcctttgattacaatggaatgtatgagtctgatcttgcgaataaacgtaaagataactcgtatcgttatttcaataacatcaaccgtctagccaaggagtttcccaaggcacatcgccagaccgaagatgacaaggtgaccgtctggtgctctaacgactacttaggaatgggtaggcatcctgagattatcaaaaccatgaaggctaccatggacaagtacggttccggagcaggaggaactaggaacattgcaggtcataaccacgccgctatcaatttggaaagcgagttggcttgcttgaacaagaaggaagcggctctggtgttttcatcatgtttcatagctaacgatgcaatcatctcgttgttgggacaaaaaatcaaaaatttggtcattttctctgaccagtcgaatcatgcttccatgatattgggtgtgcgtaactccaaagcgaagaagcacatcttcaagcacaacaatttgaaggatctggagtcgcagttagctcagtaccccaagtcgactcctaaactgatcgccttcgagtcagtttactctatgtgtggatctgtggctcccattgagaagatttgcgatttggctaaaaggtacggtgccctcaccttcttggatgaagttcatgctgttggaatgtatggtcctcatggacagggtgtagctgagcatttggactttgatctgcatttacagtctggaatcgccagtcctagcgtggtggacaaacgcaccatattggatcgtgtcgacatgattactggtacttgcggaaagtcatttggtactgttggaggttacgttgctggtagtgccaacctaattgattggttaagatcctatgcgccaggtttcattttcactaccacacttcctcctgctatcatggctggtacagccacttctgttcgtattgttagggccgacattgaggcccgtatcaagcaacagcttaatactcgctacgtcaaagactcatttgaaaaccttggtattccagtcattccaaacccaagtcacattgttcctgttctagttggaaatgctgcagatgccaagaaggcatccgatatgttaatgaacaaacaccgtatttatgttcaagctattaactaccctactgtgcctgtcggtgaagaacgactaaggattactcctactccaggtcatggaaaggagatttgtgaccagctgatcagcgctgtcgacgatgtttttactgagcttaatttaccaagaatcaacaaatggcagtcccaaggtggtcattgcggtgttggtgatgctaattacgtaccagaacccaatctgtggactcaggaccagctcagcttgacaaaccaagacttgcactccaatgtgcacaacccagtgattgagcagatcgaaacctcatcaggagtcagattgtag

SEQ ID NO: 31 - protein, 560 aa, synthetic construct
MEFVARQSMNASPFVRSTSTHHLKKLAANSSLAATASHSPVVGPALQQQRYYSQPSKPAQAQTSDIATGIKKDVSPIRMDSNETAFDYNGMYESDLANKRKDNSYRYFNNINRLAKEFPKAHRQTEDDKVTVWCSNDYLGMGRHPEIIKTMKATMDKYGSGAGGTRNIAGHNHAAINLESELACLNKKEAALVFSSCFIANDAIISLLGQKIKNLVIFSDQSNHASMILGVRNSKAKKHIFKHNNLKDLESQLAQYPKSTPKLIAFESVYSMCGSVAPIEKICDLAKRYGALTFLDEVHAVGMYGPHGQGVAEHLDFDLHLQSGIASPSVVDKRTILDRVDMITGTCGKSFGTVGGYVAGSANLIDWLRSYAPGFIFTTTLPPAIMAGTATSVRIVRADIEARIKQQLNTRYVKDSFENLGIPVIPNPSHIVPVLVGNAADAKKASDMLMNKHRIYVQAINYPTVPVGEERLRITPTPGHGKEICDQLISAVDDVFTELNLPRINKWQSQGGHCGVGDANYVPEPNLWTQDQLSLTNQDLHSNVHNPVIEQIETSSGVRL

SEQ ID NO: 32 - skipped

SEQ ID NO: 33 - protein, 5 aa, synthetic construct
ACPFV

SEQ ID NO: 34 - protein, 5 aa, synthetic construct
HCPVV

SEQ ID NO: 35 - protein, 5 aa, synthetic construct
ICPFM

SEQ ID NO: 36 - protein, 5 aa, synthetic construct
GCPVV

SEQ ID NO: 37 - DNA, 41 bp, synthetic construct
  source 1..41: mol_type = other DNA
cgtcagtccatgaatgcctctccctttgtcaggtcaacttc

SEQ ID NO: 38 - DNA, 41 bp, synthetic construct
  source 1..41: mol_type = other DNA
gaagttgacctgacaaagggagaggcattcatggactgacg

SEQ ID NO: 39 - DNA, 37 bp, synthetic construct
  source 1..37: mol_type = other DNA
gctgctactgctagtcattctcccgtggttggccctg

SEQ ID NO: 40 - DNA, 37 bp, synthetic construct
  source 1..37: mol_type = other DNA
cagggccaaccacgggagaatgactagcagtagcagc

SEQ ID NO: 41 - DNA, 27 bp, synthetic construct
  source 1..27: mol_type = other DNA
aaacgctgtcttggaacctaatatgac

SEQ ID NO: 42 - DNA, 38 bp, synthetic construct
  source 1..38: mol_type = other DNA
gacgggcgacaaactccatcgtttcgaataattagttg

SEQ ID NO: 43 - DNA, 40 bp, synthetic construct
  source 1..40: mol_type = other DNA
caactaattattcgaaacgatggagtttgtcgcccgtcag

SEQ ID NO: 44 - DNA, 45 bp, synthetic construct
  source 1..45: mol_type = other DNA
aattaaatacatttcaactacaatctgactcctgatgaggtttcg

SEQ ID NO: 45 - DNA, 42 bp, synthetic construct
  source 1..42: mol_type = other DNA
cctcatcaggagtcagattgtagttgaaatgtatttaatttg

SEQ ID NO: 46 - DNA, 24 bp, synthetic construct
  source 1..24: mol_type = other DNA
aaactgtcagttttgggccatttg

SEQ ID NO: 47 - DNA, 21 bp, synthetic construct
  source 1..21: mol_type = other DNA
gctctccaacagcagagatac

SEQ ID NO: 48 - DNA, 21 bp, synthetic construct
  source 1..21: mol_type = other DNA
gtccatacggatcggagaaac

SEQ ID NO: 49 - DNA, 19 bp, synthetic construct
  source 1..19: mol_type = other DNA
agcaacatccctgattccg

SEQ ID NO: 50 - DNA, 20 bp, synthetic construct
  source 1..20: mol_type = other DNA
atgcgtaccttcaatcctgg

SEQ ID NO: 51 - DNA, 24 bp, synthetic construct
  source 1..24: mol_type = other DNA
aagcccaaacctccgacattgcta

SEQ ID NO: 52 - DNA, 28 bp, synthetic construct
  source 1..28: mol_type = other DNA
tcgccgtaagttcttggtttagacgttc

SEQ ID NO: 53 - DNA, 23 bp, synthetic construct
  source 1..23: mol_type = other DNA
tagcgcagtctctctatcgcttc

SEQ ID NO: 54 - DNA, 20 bp, synthetic construct
  source 1..20: mol_type = other DNA
cactgggttgtgcacattgg

SEQ ID NO: 55 - DNA, 20 bp, synthetic construct
  source 1..20: mol_type = other DNA
acaatattcttctctgccgc

SEQ ID NO: 56 - DNA, 20 bp, synthetic construct
  source 1..20: mol_type = other DNA
ttgatctcgtcaagaatgcg

SEQ ID NO: 57 - DNA, 23 bp, synthetic construct
  source 1..23: mol_type = other DNA
taggtgccacaacttttggtttc

SEQ ID NO: 58 - DNA, 24 bp, synthetic construct
  source 1..24: mol_type = other DNA
gatccaatgcgatgacattcttgt

SEQ ID NO: 59 - DNA, 24 bp, synthetic construct
  source 1..24: mol_type = other DNA
acctgcaataactcctcttctctg

SEQ ID NO: 60 - DNA, 20 bp, synthetic construct
  source 1..20: mol_type = other DNA
ccactgagggtagccgaatc

SEQ ID NO: 61 - DNA, 22 bp, synthetic construct
  source 1..22: mol_type = other DNA
gggctctgaaaaactcttttgg

SEQ ID NO: 62 - DNA, 27 bp, synthetic construct
  source 1..27: mol_type = other DNA
gcatgtctcaataacagatctcgacgg

SEQ ID NO: 63 - DNA, 27 bp, synthetic construct
  source 1..27: mol_type = other DNA
aagcctcttgtttttctgtaaatgcac

SEQ ID NO: 64 - DNA, 22 bp, synthetic construct
  source 1..22: mol_type = other DNA
tgatggcgtccgagatgaactc

SEQ ID NO: 65 - DNA, 21 bp, synthetic construct
  source 1..21: mol_type = other DNA
atggagtttgtcgcccgtcag

SEQ ID NO: 66 - DNA, 27 bp, synthetic construct
  source 1..27: mol_type = other DNA
ctacaatctgactcctgatgaggtttc